(19) United States
(12) Patent Application Publication
(10) Pub. No.: US 2024/0412813 A1
(43) Pub. Date: Dec. 12, 2024

(54) METHODS AND SYSTEMS FOR TUMOUR MONITORING

(71) Applicant: Cancer Research Technology Limited, London (GB)

(72) Inventors: Alexander Mark Frankell, London (GB); Christopher Abbosh, London (GB); Robert Charles Swanton, London (GB)

(73) Assignee: Cancer Research Technology Limited, London (GB)

(21) Appl. No.: 18/699,186

(22) PCT Filed: Oct. 7, 2022
(86) PCT No.: PCT/EP2022/077987

§ 371 Date: Apr. 5, 2024

(30) Foreign Application Priority Data
Oct. 8, 2021 (GB) 2114434.0

(51) Int. Cl. G16B 20/10; C12Q 1/6886; G16B 20/20

(52) U.S. Cl. CPC G16B 20/10; C12Q 1/6886; G16B 20/20; C12Q 2600/156

(57) ABSTRACT

The present invention provides a computer-implemented method for estimating the cancer cell fraction (CCF) of at least one tumour-specific mutation in a subject. Also provided are related methods for monitoring the clonal dynamics of a tumour, monitoring a treatment of the tumour and methods for treating a subject having a cancer, as well as systems for implementing the methods of the invention.

This application claims priority from GB2114434.0 filed 8 Oct. 2021, the contents and elements of which are herein incorporated by reference for all purposes.

### FIELD OF THE INVENTION

The present invention relates to cancer detection and monitoring and particularly, although not exclusively, to methods for estimating and/or quantifying the cancer cell fraction (CCF) of cells in a tumour that harbour a particular genomic event. Determining the clonal architecture of a tumour through time facilitates the tracking of therapeutic resistance through time and allows for accurate identification of events that are clonal or become clonal at relapse which therefore become optimal therapeutic targets.

### BACKGROUND

Outgrowth of resistant cancer cell populations is a common mechanism of therapy failure in oncology. Effective personalised medicine is reliant on targeting aberrations that are present in every tumour cell. However, tumours are heterogenous and comprehensive tumour tissue sampling is often impossible. Even where multiregional sampling of solid tumours is possible, significant sampling bias may be present because spatially restricted clones may be over-sampled or under-sampled. Liquid biopsies have the potential to provide representative tumour sampling at regular intervals through disease course, but current clonal deconvolution methods are ineffective in low tumour content samples (<5%) which comprise most samples in the localised or minimal residual disease (MRD) settings.

A variety of algorithms have been developed to reconstruct the subclonal architectures of cancers from single-region or multi-region bulk DNA sequencing data (see, for example, Liu, L. Y., Bhandari, V., Salcedo, A. et al. Quantifying the influence of mutation detection on tumour subclonal reconstruction. Nat Commun 11, 6247 (2020)). Absolute quantification of somatic DNA alterations in human cancer and the ABSOLUTE algorithm are described in Carter et al., Nature Biotechnology, 2012, Vol. 30, No. 5, pp. 413-421.

US2020/0248266 describes subject-specific methods for detecting recurrence of tumours based on an understanding of the clonal/subclonal mutation profile of the subject's tumour and detection of the mutations in their cell-free DNA (cfDNA), typically by multiplex PCR of tumour mutations such as single nucleotide variants (SNVs).

Frankell et al., 2022, [abstract]. In: Proceedings of the American Association for Cancer Research Annual Meeting 2022; 2022 Apr. 8-13. Philadelphia (PA): AACR; Cancer Res 2022; 82 (12_Suppl): Abstract nr 2144, describe holistic sampling of clonal dynamics using cfDNA in lung TRACERx.

Methods for determining the clonal architecture of tumours using tissue sequencing have been developed over the past 10 years. However, these methods are not usually applicable to circulating tumour DNA (ctDNA) samples due to their ultra-low purity, i.e. only a very small percentage of DNA in these samples is derived from the tumour, usually <1%. This precludes accurate calling of copy number events, where whole chromosomes or parts of chromosomes have been amplified or deleted, which usually requires a cellularity of at least 10% even with deep whole exome or whole genome sequencing and is essential in most carcinomas which have large numbers of these events to accurately estimate the percentage of cells in a tumour which harbour a genomic event (i.e. the CCF). Mutations can be detected in ultra-low cellularity samples using targeted ultra-deep sequencing of genomic positions known to be mutated. Ultra-low purity therefore makes standard clonality extraction methods inapplicable or highly inaccurate in the vast majority of ctDNA samples.

The present invention has been devised in light of the above considerations. The present invention aims to mitigate problems with prior methods of tumour monitoring and of CCF estimation, in particular, as well as providing certain related advantages.

### SUMMARY OF THE INVENTION

The present inventors have developed a method which takes estimates of the copy number state at each mutated position and the clonal groups to which each mutation belongs from matched tumour tissue sequencing and uses this information with a reconfiguration of the equations from tissue clonality extraction methods and estimates of background sequencing noise to accurately deconvolve the clonal architecture. As described in detail herein, the resulting estimates of cancer cell fraction (CCF) are able to provide actionable insights into sub-clonal tumour dynamics relevant to treatment and prognosis of cancer. In some embodiments, these estimates of CCF are superior to those obtained using tissue sampling alone as they are less prone to sampling bias.

Accordingly, in a first aspect the present invention provides a computer-implemented method for estimating the cancer cell fraction (CCF) of at least one tumour-specific mutation in a subject, the method comprising:
(i) providing sequence data obtained from a sample comprising cell-free DNA, which includes circulating tumour DNA (ctDNA) from the subject, the sequence data comprising: the variant allele fraction (VAF), being equal to the total number of reads in the sample that show the tumour-specific mutation divided by the total number of reads (mutated and germline) at the location of the tumour-specific mutation;
(ii) providing sequence data obtained from a sample comprising DNA obtained from tumour tissue of the subject, the sequence data comprising: the multiplicity of said at least one tumour-specific mutation; and the copy number at the location of the tumour-specific mutation (CNₜᵤₘₒᵤᵣ);
(iii) providing the germline copy number at the location of the tumour-specific mutation (CNₙₒᵣₘₐₗ);
(iv) providing an estimate of the purity of said sample comprising cell-free DNA, the purity being the proportion of cells contributing to the sampled DNA which are tumour cells; and
(v) determining the estimate of CCF for the at least one tumour-specific mutation according to the formula:

$$CCF = VAF \frac{1}{multiplicity * Purity} ( Purity \times {CN}_{tumour} + ( 1 - Purity ) \times {CN}_{normal} )$$

wherein VAF is as provided in (i), multiplicity and CNₜᵤₘₒᵤᵣ are as provided in (ii), CNₙₒᵣₘₐₗ is as provided in (iii) and purity is as provided in (iv).

In some embodiments, providing an estimate of purity of said sample comprising cell-free DNA comprises:
providing, for each of a plurality of further tumour-specific mutations that have previously been determined to be clonal mutations, the VAF of the further mutation in said sample comprising cell-free DNA, the multiplicity of the further mutation, the CNₜᵤₘₒᵤᵣ of the further mutation, and the CNₙₒᵣₘₐₗ at the location of the further mutation;
determining the mutation-specific purity of each of said plurality of further mutations according to the formula:

$$Purity = \frac{{CN}_{normal}}{\frac{multiplicity}{VAF} - {CN}_{tumour} + {CN}_{normal}}$$

and
estimating the purity of said sample by combining (e.g. averaging) the mutation-specific purity values of each of said plurality of further mutations.

In some cases, the at least one tumour-specific mutation comprises at least 2, 3, 4 or at least 5 tumour-specific mutations belonging to a single sub-clonal population of tumour cells.

In some cases, the estimated CCF for each of said tumour-specific mutations belonging to the single sub-clonal population are combined (e.g. averaged) to provide a CCF estimate for the sub-clonal population of tumour cells.

The order of the steps and the spacing apart in time of the steps of the methods of the present invention are not particularly limited. For example, it is specifically contemplated that the tumour tissue sample and the cell free DNA sample may be taken at the same time. This may have particular benefit in that the ECLIPSE method of the present invention may improve the reliability of a determination of clonality made for a mutation or a cancer cell population derived solely from a tumour tissue sample. In some cases, the cell-free DNA sample (e.g. plasma sample) may be taken at a later time point (e.g. hours, days, months or even years later) as compared with the tumour tissue sample. It is specifically contemplated that multiple cell-free DNA samples may be taken a different time points, for example as part of the monitoring of a cancer especially in the context of cancer treatment.

In some cases, a correction for the background sequencing error is applied to estimate whether the number of reads in the sample that show the tumour-specific mutations from a given sub-clonal population of cells is likely to be genuine or due to sequencing error. This may involve applying a statistical test to compare (i) the total number of reads in the sample that show the tumour-specific mutations from the sub-clonal population and (ii) the background sequencing error rate at the location of each of the tumour-specific mutations multiplied by the total number of reads at the location of each of the tumour-specific mutations. The statistical test may be used to determine whether the particular sub-clonal population is present in the sample or not, For example, if the p-value of the statistical test is greater than 0.05, the sub-clonal population of cells may be considered not to be present in the sample. In some cases the statistical test may be selected from the group consisting of: a binomial test, a Poisson test, a one sample Wilcoxon rank sum test (using an expected background distribution), a chi-squared/Fisher's exact test (comparing the expected reference and variant counts to the observed reference and variant counts).

In some cases, the sample comprising DNA obtained from tumour tissue of the subject is obtained at an earlier point in time than the sample comprising cell-free DNA. In particular, a tissue biopsy may be taken and then followed up by one or more liquid biopsies and the method of the present invention applied to track changes in CCF of particular mutations and/or tumour clonal populations over time.

In some cases, the sequence data has been obtained from multiple samples comprising cell-free DNA, which includes circulating tumour DNA (ctDNA), from the subject at different time points. In particular, the different time points may be different time points during a course of treatment of the tumour.

In some cases, the sample comprising cell-free DNA may be a liquid sample, such as a plasma sample, a blood sample, a urine sample or a cerebrospinal fluid (CSF) sample.

In some cases, the purity of the or each sample comprising cell-free DNA may be 5% or lower, such as 4%, 3%, 2%, 1% or 0.5% or lower. The present inventors have found that the ECLIPSE method of the present invention may, in some embodiments, permit reliable estimation of CCF even in low purity samples such as those frequently encountered in the context of minimal residual disease (MRD) and post-surgery cancer treatment.

In some cases, the at least one tumour-specific mutation gives rise to a suspected or known neoantigen and/or give rise to a target for an anti-cancer therapy. As the skilled person will appreciate a number of anti-cancer agents are for use with or show superior results when the patient being treated has a particular mutation (e.g. EGFR mutation).

In some cases the method further comprises providing to a user the determined CCF of at least one tumour-specific mutation and/or at least one clonal or sub-clonal tumour cell population. This may involve displaying the determined CCF (e.g. the fraction or decimal number or other indication of CCF or degree of clonality) on a user interface or transmitting to the user, e.g., via a network.

In a second aspect, the present invention provides a method for estimating the cancer cell fraction (CCF) of at least one tumour-specific mutation in a subject, the method comprising:
providing a cfDNA-containing sample, which sample includes ctDNA, obtained from the subject;
sequencing DNA from said cfDNA-containing sample or from a library prepared from said cfDNA-containing sample to produce sequence data; and
performing the method of the first aspect of the invention using said sequence data and thereby estimating the CCF of the at least one tumour-specific mutation in the subject.

In some cases, the method further comprises:
providing a sample comprising DNA obtained from tumour tissue of the subject;
sequencing DNA from said sample comprising DNA obtained from tumour tissue or from a library prepared from said sample comprising DNA obtained from tumour tissue to produce tumour tissue sequence data; and
analysing the tumour tissue sequence data produced to determine the multiplicity of said at least one tumour-specific mutation; and the copy number at the location of the tumour-specific mutation (CNₜᵤₘₒᵤᵣ).

In a third aspect, the present invention provides a method for identifying at least one tumour-specific mutation, or a population of tumour cells harbouring said at least one tumour-specific mutation, in a subject as a potential therapeutic target, the method comprising:
performing the method of the first aspect of the invention at least once to estimate the CCF of the at least one tumour-specific mutation or the population of cells harbouring said at least one tumour-specific mutation; and
selecting the at least one tumour-specific mutation or the population of cells harbouring said at least one tumour-specific mutation as a potential therapeutic target, provided that at least one of the following is true:
the CCF is estimated to be at least 0.3, 0.4, 0.5, 0.6, 0.7, 0.8, 0.9 or at least 0.95;
the CCF is estimated at at least two different time points and is found to be rising; and
the CCF is estimated before and after a treatment intervention for said tumour and the CCF is found to be declining following said treatment intervention.

In a fourth aspect, the present invention provides a method for monitoring the clonal dynamics of a tumour and/or monitoring a treatment of the tumour, the method comprising:
performing the method of the first aspect of the invention to estimate the CCF of the at least one tumour-specific mutation or the population of cells harbouring said at least one tumour-specific mutation at two or more time points for the same subject; and
tracking the estimated CCF at said two or more time points to monitor change in the CCF over time.

In some cases, the CCF of at least 2, 3, 4, 5, 6, 7, 8, 9, 10 or at least 20 tumour-specific mutations and/or the CCF of at least 2, 3, 4, 5, 6, 7, 8, 9, 10 or at least 20 clonally distinct populations of cells of said tumour are estimated.

In some cases in accordance with any aspect of the present invention, the at least one tumour-specific mutation is selected from the group consisting of: a single nucleotide variant (SNV), a multiple nucleotide variant (MNV), a deletion mutation, an insertion mutation, an indel mutation, a translocation, a missense mutation, a translocation, a fusion, a splice site mutation, or any other change in the genetic material of a tumour cell. Without wishing to be bound by any particular theory, the present inventors believe that the ECLIPSE method of the present invention is applicable to any genetic alterations where VAF and copy number can be measured with some accuracy. These specifically include SNVs, multi-nucleotide variants, small insertions/deletions and structural variants. In certain embodiments, as described in detail herein, the at least one tumour-specific mutation is a single nucleotide variant.

In some cases, the at least one tumour-specific mutation results in the mutated DNA encoding a neoantigen and/or wherein the at least one tumour-specific mutation is or encodes a target for an anti-cancer therapy.

In a fifth aspect, the present invention provides a method for treating a subject having a cancer, the method comprising:
performing the method of any aspect of the present invention, wherein the estimated CCF of the at least one tumour-specific mutation indicates that the tumour-specific mutation is present in the present in the tumour at a level sufficient to render the tumour-specific mutation an effective target for therapy; and
administering an anti-cancer therapy that targets said tumour-specific mutation.

In some embodiments, at least one of the following may be true:
the CCF is estimated to be at least 0.3, 0.4, 0.5, 0.6, 0.7, 0.8, 0.9 or at least 0.95;
the CCF is estimated at at least two different time points and is found to be rising; and
the CCF is estimated before and after administration of said anti-cancer therapy and the CCF is found to be declining following said administration.

In some cases, the tumour of the subject has, or is suspected of having, metastasized; the subject has had treatment aimed at surgical removal of one or more tumours; the subject has been treated with one or more anti-cancer therapeutic agents; and/or the subject has a cancer which has relapsed or the subject is suspected to be at risk of cancer relapse.

In a sixth aspect, the present invention provides a system comprising:
a processor; and
a computer readable medium comprising instructions that, when executed by the processor, cause the processor to perform the steps of the method of the first aspect of the invention.

In a seventh aspect, the present invention provides one or more computer readable media comprising instructions that, when executed by one or more processors, cause the one or more processors to perform the steps of the method of the first aspect of the invention.

In accordance with any aspect of the present invention, the subject may have any cancer. In some embodiments, the cancer may be a solid tumour (primary and/or metastatic). In some cases, the cancer may be a cancer that harbours within the genome and/or exome of at least a portion of the cancer cells at least 50, at least 75, at least 100, at least 500, at least 1000, at least 5000 or at least 10,000 cancer-specific mutations (i.e. mutations as compared with the germ-line genomic sequence of the subject as found in one or more non-cancer cells of the subject).

In some cases, the cancer may be the cancer may be lung cancer (small cell, non-small cell and mesothelioma), ovarian cancer, breast cancer, endometrial cancer, kidney cancer (renal cell), brain cancer (glioma, astrocytoma, glioblastoma), melanoma, merkel cell carcinoma, clear cell renal cell carcinoma (ccRCC), lymphoma, small bowel cancers (duodenal and jejunal), leukemia, pancreatic cancer, hepatobiliary tumours, germ cell cancers, prostate cancer, head and neck cancers, thyroid cancer and sarcomas. For example, the cancer may be lung cancer, such as lung adenocarcinoma or lung squamous-cell carcinoma. As another example, the cancer may be melanoma. In embodiments, the cancer may be selected from melanoma, merkel cell carcinoma, renal cancer, non-small cell lung cancer (NSCLC), urothelial carcinoma of the bladder (BLAC) and head and neck squamous cell carcinoma (HNSC) and microsatellite instability (MSI)-high cancers. In some embodiments, the cancer is non-small cell lung cancer (NSCLC). In other embodiments, the cancer is melanoma.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows a schematic overview of the ECLIPSE architecture.

FIG. 2 shows a schematic illustration of tissue sampling bias and its potential avoidance by plasma sampling.

FIG. 3 shows the correlation between cancer cell fractions (CCFs) measured in plasma taken before surgery (y-axis).using ECLIPSE (A) or without ECLIPSE (B) and measured using multiregional tissue sampling at surgery (x-axis). The inset shows the scale for plasma purity by dot size.

FIG. 4 (left) shows a bar graph of the percentage of clones detected in plasma split by those present in 1 region or present in more than 1 region; and (right) box plots of the number of mutations followed in plasma.

FIG. 5 shows a depiction of a schematic illustration of the “Winner's Curse” effect, whereby single region sub-clones may be over-sampled using tissue sampling.

FIG. 6 shows (left) box plots of CCF determined from plasma (using ECLIPSE) and from tissue. This shows that for sub-clones that are detected and are unique to single regions, CCF is usually found to be lower in plasma than in tissue due to the tissue sampling bias effect depicted on the right.

FIG. 7 shows CCF box plots for plasma and tissue sampling, split by tumour volume (<20 cm³, >20 cm³ and <100 cm³, or >100 cm³.

FIG. 8 shows a depiction of the ability of ECLIPSE-based plasma sampling to overcome the “clonal illusion” phenomenon.

FIG. 9 shows an example case of tracking tumour clonal dynamics with ctDNA and ECLIPSE.

FIG. 10 shows an example case of tracking tumour clonal dynamics with ctDNA and ECLIPSE, in which plasma sampling was found to detect a sub-clonal lineage not captured by tissue biopsies.

FIG. 11 shows that additional sub-clones were detected by ctDNA liquid biopsy using ECLIPSE in comparison with tissue sampling.

FIG. 12 shows that ctDNA sampling using ECLIPSE provided a more clonally complex picture (more polyclonal and polyphyletic) in comparison with tissue sampling.

FIG. 13 shows that it was possible to track clonal structure with ctDNA by ECLIPSE in the setting of adjuvant chemotherapy and minimal residual disease (MRD) depsite low ctDNA fractions found in these samples.

FIG. 14 shows KM survival curves for overall survival (left) and post-relapse survival (right) split into monoclonal (blue), polyclonal (yellow) and polyphyletic (red). Notably, more clonally complex tumours were found to be more aggressive.

FIG. 15 shows the relationship between sub-clone size (i.e. CCF as detected using plasma ctDNA samples with ECLIPSE) and metastatic potential. Sub-clones that go on to metastasize are those that were larger in the primary tumour (A) and patients that develop metastases tend to have larger sub-clones in their primary tumour.

FIG. 16 shows a further example case of tracking tumour clonal dynamics with ctDNA and ECLIPSE, in which plasma sampling was found to detect a sub-clonal lineage not captured by tissue biopsies. Treatments at the indicated timepoints post-surgery are shown.

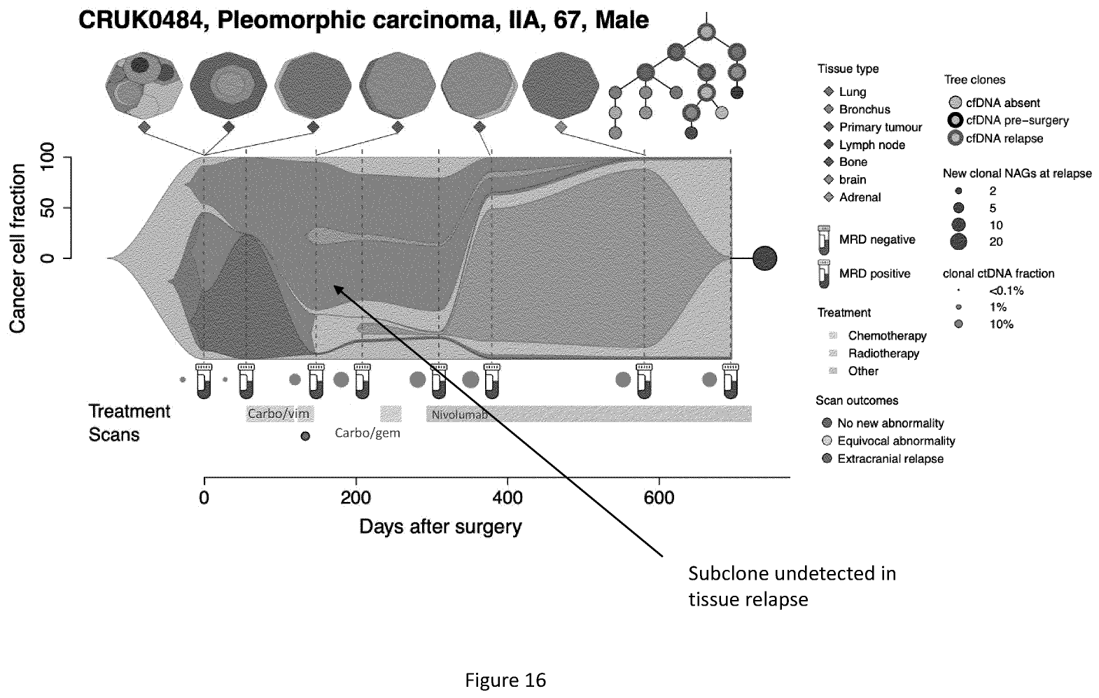

FIG. 17 shows nine examples of longitudinal evolution with different metastatic seeding patterns.

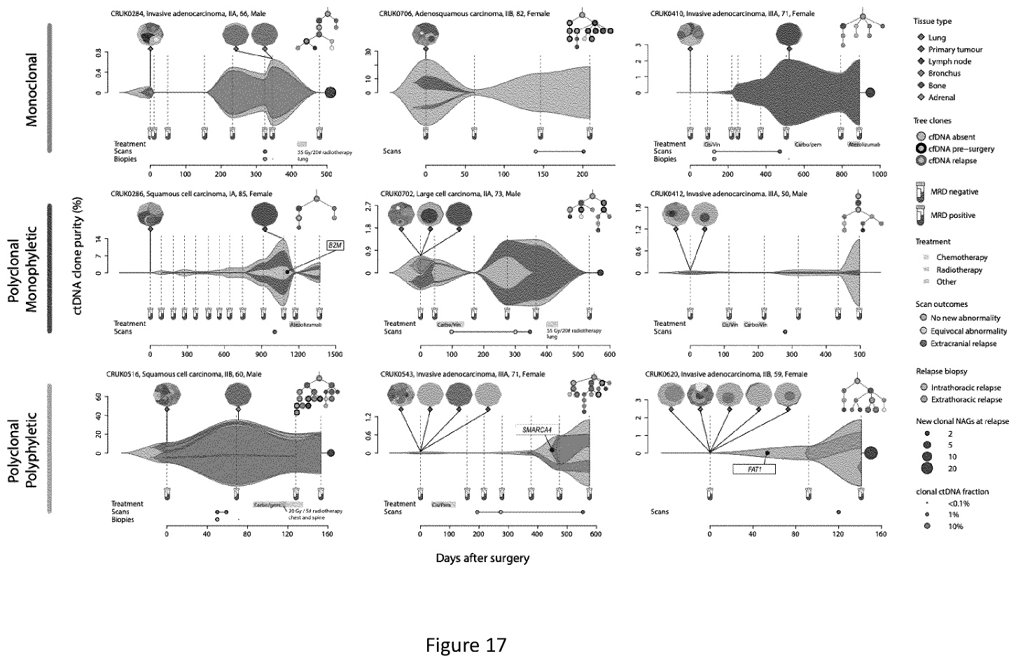

FIG. 18 shows survival analysis showing improved separation with further data (c.f. FIG. 14).

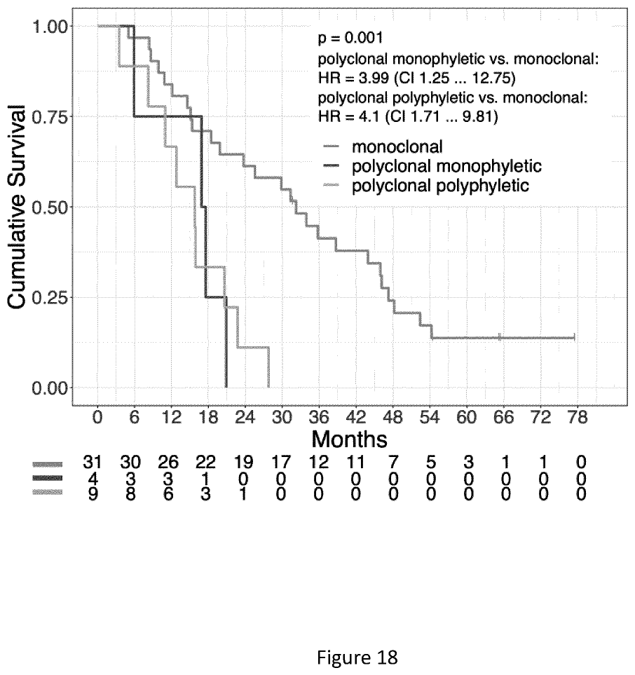

FIG. 19 shows a multivariable model of overall survival controlling clinical and other factors.

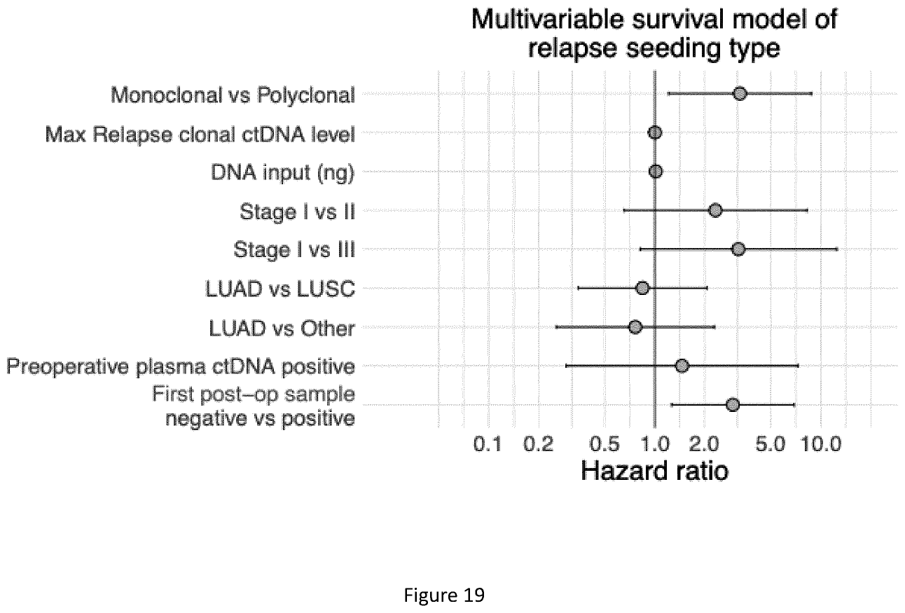

FIG. 20 shows the relationship between sub-clone size (i.e. CCF as detected using plasma ctDNA samples with ECLIPSE) and metastatic potential. The results more clearly show the difference between relapsing and non-relapsing subclones with further information (c.f. FIG. 15).

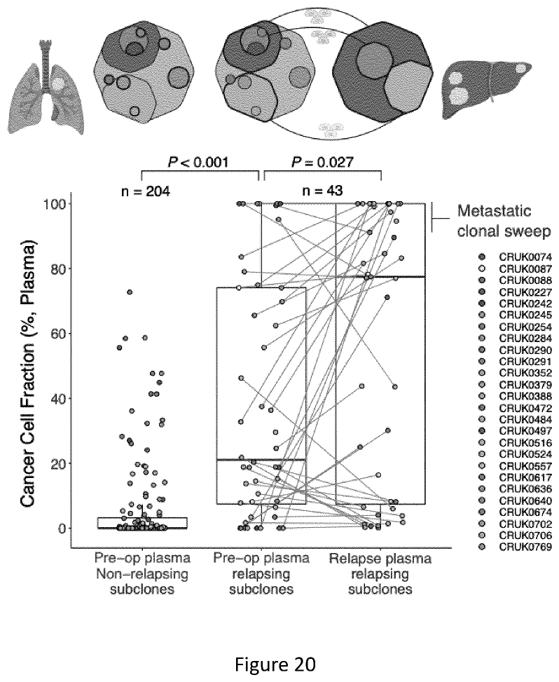

FIG. 21 shows for each sample the estimated number of mutant reads that would be required to detect an average subclone's presence given the depth of sequencing and background noise in each sample (at P=0.01). This number of reads is then converted to a Cancer cell fraction using ECLIPSE which represents the minimal cancer cell fraction which would be detectable in each sample plotted below. This suggests that at 0.1% the ECLIPSE method of the invention would be able to detect subclones at 20% Cancer cell fraction thereby providing an indication of the limit of detection (LOD) of ECLIPSE.

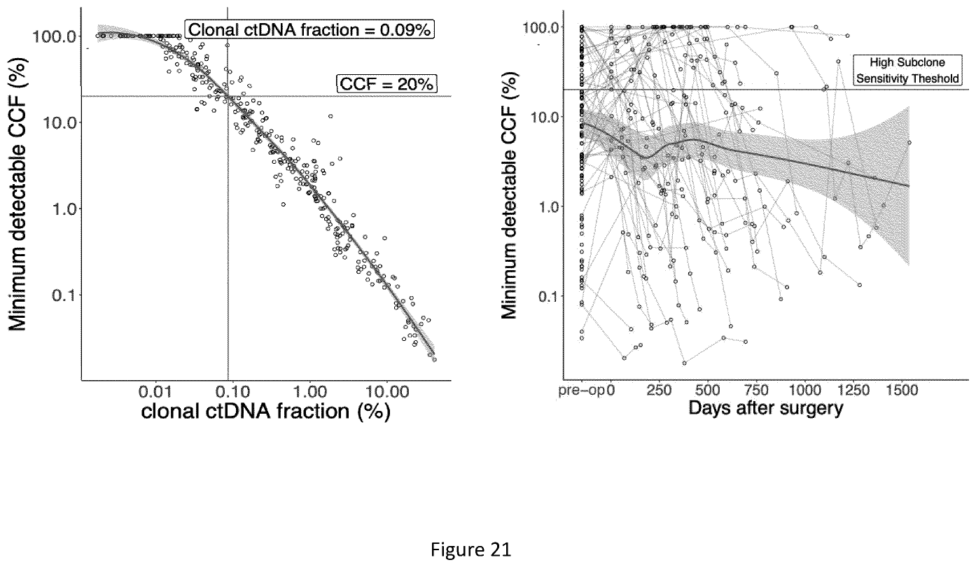

FIG. 22 shows that the ECLIPSE method of the present invention can characterise ctDNA samples of at least 0.1% ctDNA fraction whereas standard methods are limited to samples with at least 10% ctDNA fraction. In this context, “standard methods” for clonal reconstruction were considered as those used as standard for tumour tissue based clonal construction such as PyClone (doi.org/10.1038/nmeth.2883), DPClust (github.com/Wedge-lab/dpclust), DeCiFer (github.com/raphael-group/decifer) which rely on broad sequencing of either the whole exome or whole genome. These and similar methods are the only validated methods that have been applied to plasma samples to determine clonal structure and require, at minimum, tumour fractions of >10%. For example a recent paper in Nature Herberts et al 2022 of castrate resistant metastatic prostate cancer, included only patients with ctDNA samples of at least 30% tumour fraction for their whole genome sequencing based clonality analyses.

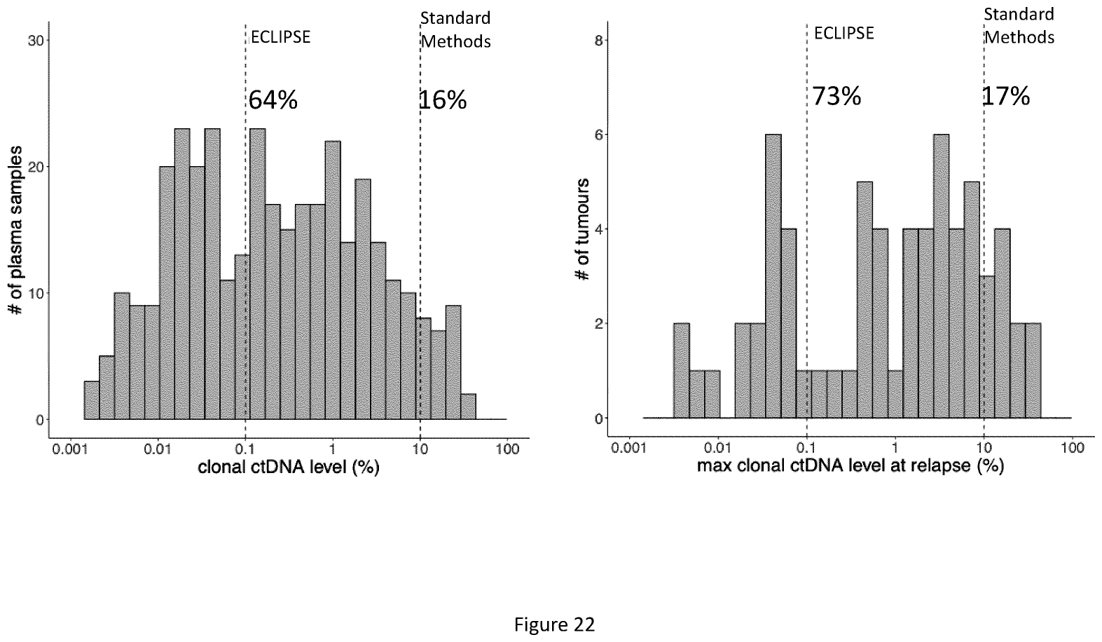

Only 16% of our ctDNA positive samples were >10% ctDNA fraction but 64% have at least 0.1% ctDNA fraction. Only 17% of TRACERx ever have a ctDNA sample at relapse of 10% ctDNA fraction whereas 73% of patients have ctDNA sample of >0.1% at relapse. Therefore, ECLIPSE enables a far greater fraction of patients to be clonally characterised at relapse using ctDNA.

FIG. 23 shows the use of the ECLIPSE method of the present invention to overcome a problem of characterizing clonal structure at relapse from relapse tissue.

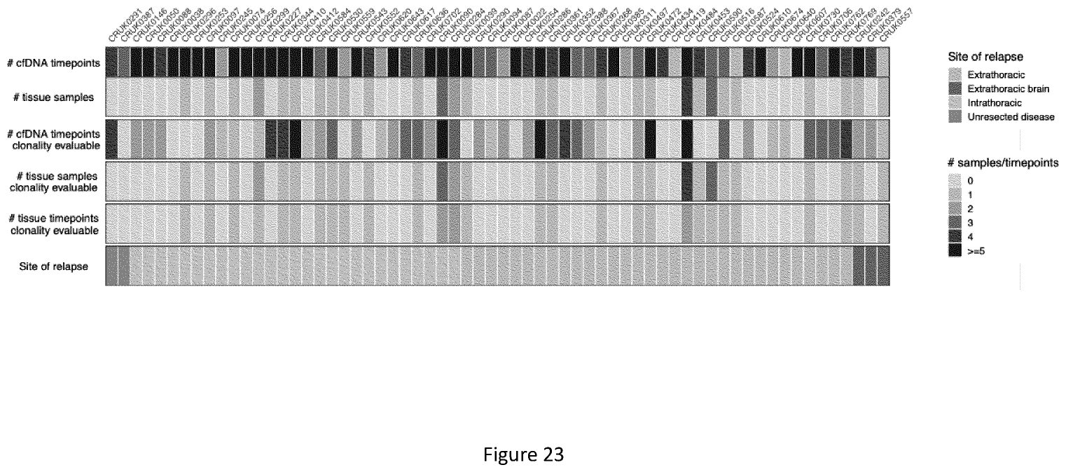

It is challenging to get a relapse tissue biopsy to characterise clonal structure at relapse. In TRACERx it is only possible to obtain a relapse biopsy in 44% of cases but by combining this with >0.1% ctDNA fraction plasma the present inventors were able to characterise 82% of cases. This shows that relapse clonality characterization can be improved by applying the ECLIPSE method of the present invention to liquid biopsy samples in order to supplement tissue biopsy.

FIG. 24 shows that ECLIPSE determined that 1/3 patients undergo a clonal bottleneck at relapse—i.e. where a subclone grows to occupy 100% of cancer cells. In these patients the clonal tumour mutational burden (TMB) increases (as previously subclonal mutations become clonal) by an on average 20%. Clonal TMB is being investigated as a marker of Immunotherapy response. These bottlenecks also increase the number of clonal neoantigens which are being investigated for therapeutic targeting in clinical trials.

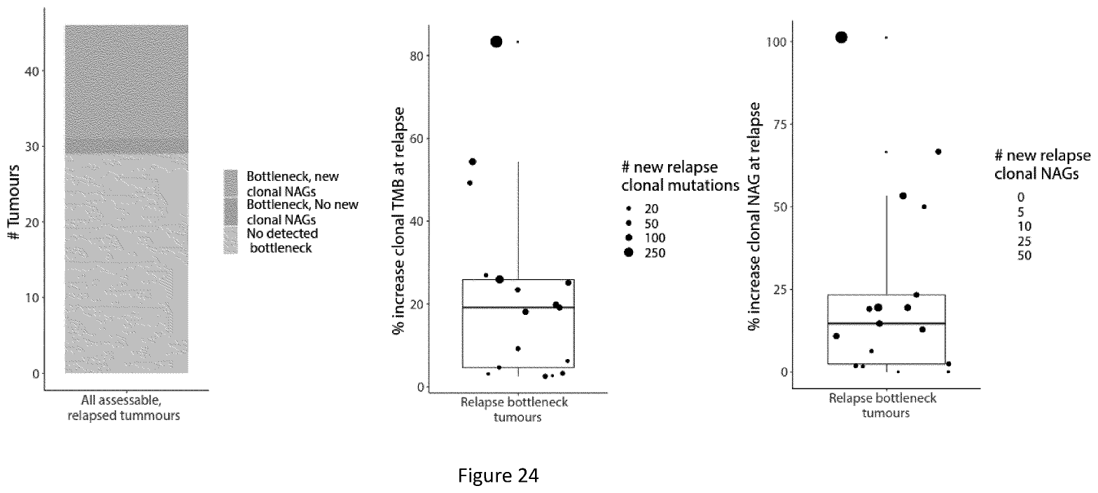

FIG. 25 shows a depiction of the ability of ECLIPSE-based plasma sampling to overcome the “clonal illusion” phenomenon. Panels (A) and (B) reproduce data shown in FIG. 8. Panel (C) is the result of additional analysis and shows an AUC of 0.81 for predicting clonal vs. clonal illusion using plasma CCF.

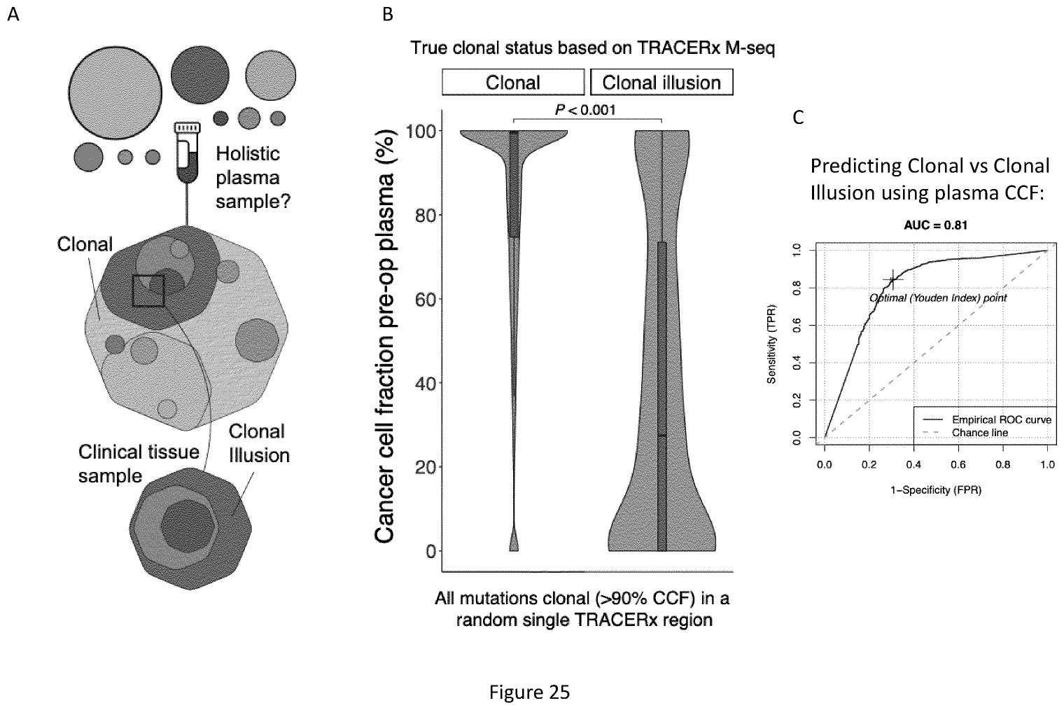

FIG. 26 shows by accounting for DNA copy number ECLIPSE can measure ctDNA tumour purity (the fraction of cells from which the cfDNA derives that are tumour cells) and in combination with the input DNA mass and plasma volume can allow estimation of the number of normal (non-tumour) and tumour genomes which were present per milliliter of plasma taken from the patient. This has never previously been estimated to our knowledge. This this more strongly correlates with ct-scan measured tumour volume that mean clonal vaf (a common measure in used in the field) in adenocarcinomas suggesting that by correcting for copy number and plasma volume we can better measure the amount tumour disease burden using ECLIPSE in adenocarcinomas.

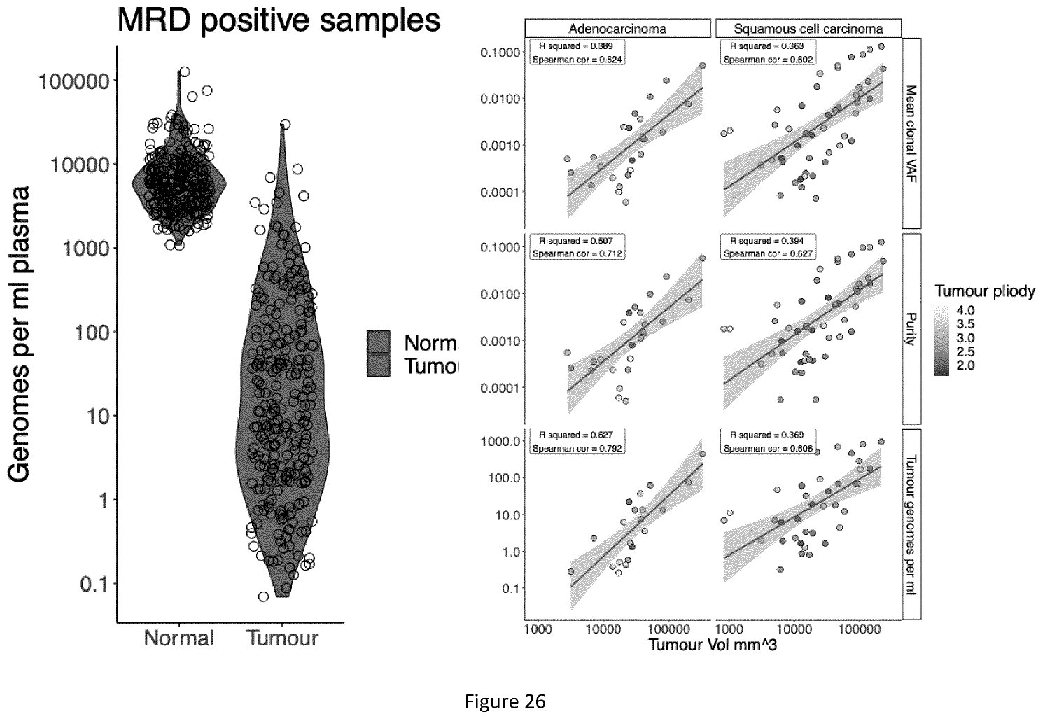

FIG. 27 shows limit of detection (LOD) estimates for subclone detection by the ECLIPSE method of the present invention across different ground truth; ctDNA fractions, nanogram DNA inputs into sequencing assay, numbers of mutations tracked per subclone and subclonal cancer cell fractions, using sequencing of contrived DNA samples with spike-in mutations at known allele frequencies.

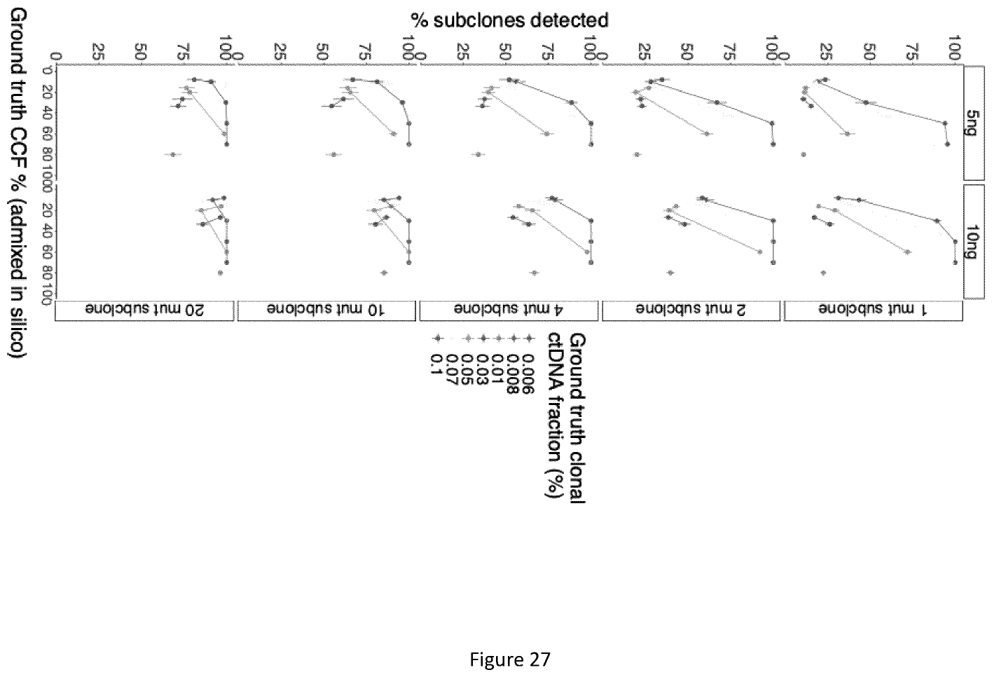

Mutations from pairs of samples with different ground truth spike in allele frequencies were combined in silico and designated as clonal and subclonal variants producing ground truth clonal ctDNA fractions and cancer cell fractions for each set of subclonal mutations representing a single subclone. We combined different mutation sets (each forming one subclone) from 398 different spike in experiments (50 mutations each) to generate 76,236 subclones based on ground truth spike in data and applied ECLIPSE to the allelic fractions observed in the sequencing of these samples and whether each subclone was detectable by ECLIPSE or not. 12 replicates were available per condition for which the % of detected subclones was computed the average and 95% confidence intervals for detection rate across replicates is calculated.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

A “sample” as used herein may be a cell or tissue sample, a biological fluid, an extract (e.g. a DNA extract obtained from the subject), from which genomic material can be obtained for genomic analysis, such as genomic sequencing (e.g. whole genome sequencing, whole exome sequencing). The sample may be a cell, tissue or biological fluid sample obtained from a subject (e.g. a biopsy). Such samples may be referred to as “subject samples”. In particular, the sample may be a blood sample, or a tumour sample, or a sample derived therefrom. The sample may be one which has been freshly obtained from a subject or may be one which has been processed and/or stored prior to genomic analysis (e.g. frozen, fixed or subjected to one or more purification, enrichment or extraction steps). The sample may be a cell or tissue culture sample. As such, a sample as described herein may refer to any type of sample comprising cells or genomic material derived therefrom, whether from a biological sample obtained from a subject, or from a sample obtained from e.g. a cell line. In embodiments, the sample is a sample obtained from a subject, such as a human subject. The sample is preferably from a mammalian (such as e.g. a mammalian cell sample or a sample from a mammalian subject, such as a cat, dog, horse, donkey, sheep, pig, goat, cow, mouse, rat, rabbit or guinea pig), preferably from a human (such as e.g. a human cell sample or a sample from a human subject). Further, the sample may be transported and/or stored, and collection may take place at a location remote from the genomic sequence data acquisition (e.g. sequencing) location, and/or any computer-implemented method steps described herein may take place at a location remote from the sample collection location and/or remote from the genomic data acquisition (e.g. sequencing) location (e.g. the computer-implemented method steps may be performed by means of a networked computer, such as by means of a “cloud” provider).

The subject may have a cancer which comprises a solid tumour (primary and/or metastatic). In some cases, the cancer may be a cancer that harbours within the genome and/or exome of at least a portion of the cancer cells at least 50, at least 75, at least 100, at least 500, at least 1000, at least 5000 or at least 10,000 cancer-specific mutations (i.e. mutations as compared with the germ-line genomic sequence of the subject as found in one or more non-cancer cells of the subject). In some cases, the cancer may be the cancer may be selected from: lung cancer (small cell, non-small cell and mesothelioma), ovarian cancer, breast cancer, endometrial cancer, kidney cancer (renal cell), brain cancer (glioma, astrocytoma, glioblastoma), melanoma, merkel cell carcinoma, clear cell renal cell carcinoma (ccRCC), lymphoma, small bowel cancers (duodenal and jejunal), leukemia, pancreatic cancer, hepatobiliary tumours, germ cell cancers, prostate cancer, head and neck cancers, thyroid cancer and sarcomas. For example, the cancer may be lung cancer, such as lung adenocarcinoma or lung squamous-cell carcinoma. As another example, the cancer may be melanoma. In embodiments, the cancer may be selected from melanoma, merkel cell carcinoma, renal cancer, non-small cell lung cancer (NSCLC), urothelial carcinoma of the bladder (BLAC) and head and neck squamous cell carcinoma (HNSC) and microsatellite instability (MSI)-high cancers. In some embodiments, the cancer is non-small cell lung cancer (NSCLC). In other embodiments, the cancer is melanoma.

A “mixed sample” refers to a sample that is assumed to comprise multiple cell types or genetic material derived from multiple cell types. Within the context of the present disclosure, a mixed sample is typically one that comprises tumour cells, or is assumed (expected) to comprise tumour cells, or genetic material derived from tumour cells. Samples obtained from subjects, such as e.g. tumour samples, are typically mixed samples (unless they are subject to one or more purification and/or separation steps). Typically, the sample comprises tumour cells and at least one other cell type (and/or genetic material derived therefrom). For example, the mixed sample may be a tumour sample. A “tumour sample” refers to a sample derived from or obtained from a tumour. Such samples may comprise tumour cells and normal (non-tumour) cells. The normal cells may comprise immune cells (such as e.g. lymphocytes), and/or other normal (non-tumour) cells. The lymphocytes in such mixed samples may be referred to as “tumour-infiltrating lymphocytes” (TIL). A tumour may be a solid tumour or a non-solid or haematological tumour. A tumour sample may be a primary tumour sample, tumour-associated lymph node sample, or a sample from a metastatic site from the subject. A sample comprising tumour cells or genetic material derived from tumour cells may be a bodily fluid sample. Thus, the genetic material derived from tumour cells may be circulating tumour DNA or tumour DNA in exosomes. Instead or in addition to this, the sample may comprise circulating tumour cells. A mixed sample may be a sample of cells, tissue or bodily fluid that has been processed to extract genetic material. Methods for extracting genetic material from biological samples are known in the art. A mixed sample may have been subject to one or more processing steps that may modify the proportion of the multiple cell types or genetic material derived from the multiple cell types in the sample. For example, a mixed sample comprising tumour cells may have been processed to enrich the sample in tumour cells. Thus, a sample of purified tumour cells may be referred to as a “mixed sample” on the basis that small amounts of other types of cells may be present, even if the sample may be assumed, for a particular purpose, to be pure (i.e. to have a tumour fraction of 1 or 100%).

As used herein, the term “tissue sample” or “sample comprising DNA obtained from tumour tissue” may refer to a sample obtained directly from the tumour tissue, such as a tissue biopsy in which one or more cells or portions of cellular material are extracted from a tumour, or a sample obtained indirectly form the tumour tissue, such as a cell-free DNA sample (e.g. a plasma sample) containing ctDNA. As will be appreciated by the skilled person, in some cases, particularly when the purity of the cell-free DNA sample is low, it will be desirable or even necessary for the tissue sample to be obtained directly from the tumour. However, in cases where purity is high (e.g. >10%), such as when a tumour is large and/or in types of cancer known to shed higher amounts of ctDNA, it will be possible to identify reliably cancer-specific mutations and cancer-specific somatic copy number alterations from a cell-free DNA sample. In such embodiments, the cell-free DNA sample (e.g. plasma sample) containing ctDNA at a relatively high purity (e.g. >10%) may be sequenced, for example, to obtain a whole exome or whole genome sequence at sufficient depth to perform variant calling, copy number determination and/or to assign cancer-specific mutations to particular cancer cell clonal populations. Therefore in accordance with the present invention, those steps that refer to a “sample comprising DNA obtained from tumour tissue” may be taken to refer to determinations of, e.g., CNₜᵤₘₒᵤᵣ or multiplicity, made with respect to a cell-free DNA sample (e.g. a plasma sample containing ctDNA) of appropriately high purity (e.g. >5% or >10%). Those steps that refer to sequence data obtained from a sample comprising cell-free DNA, which includes circulating tumour DNA (ctDNA) from the subject, and the corresponding determinations (e.g. the variant allele fraction (VAF)) may be derived from the same cell-free DNA sample or a different cell-free DNA sample (such as a later sample or a plurality of cell-free DNA samples). In this way, “base line” information about the tumour (including, for example, the identity of a plurality of cancer-specific mutations, and their corresponding CNₜᵤₘₒᵤᵣ and multiplicity values) may be obtained from a direct tumour tissue sample or a plasma sample of sufficiently high purity. The characteristics and clonal dynamics of the tumour may then be tracked and/or monitored over time using plasma samples of lower purity (<5%) and the ECLIPSE method of the present invention, in particular to estimate CCF of particular mutations and subclonal populations of cancer cells, to identify the loss of a particular mutation or subclonal population and/or to identify that a particular mutation or set of mutations harboured by a previously subclonal population have become clonal, i.e. with a CCF statistically indistinguishable from 100%. A clonal mutation may be identified as partially or completely lost if its CCF falls below a certain threshold depending on the noise observed in the CCF data. This threshold could be as high as 0.8 in data with very little noise or as low as 0.2 in data with high amounts of noise to retain a high specificity. Moreover, a mutation or clone previously estimated to be in only a subset of tumour cells (i.e. being subclonal) may expand and become dominant across the tumour, in 100% or nearly 100% of cells (i.e. clonal). This may be detected using the CCFs estimated by ECLIPSE if a mutation's CCF or the CCFs of a collection of mutations in a clone are not distinguishable with from 100% CCF or from CCFs in clonal mutations. Specifically, a Wilcoxon test may be performed to compare CCFs of a given subclone with the CCFs from mutations estimated to be clonal. If the resulting P value is above a chosen threshold, e.g., is >0.05 and the average CCF of this subclone is greater than 0.8 we may estimate that the mutations in such a subclone have most likely become clonal in 100% or near to 100% of cells, making these mutations more attractive therapeutic targets, for example, if they are determined to be neoantigens or other otherwise therapeutically actionable (e.g. EGFR mutations). In this way the ECLIPSE method of the present invention may provide, in certain embodiments, a relatively non-invasive way to track the clonal dynamics and evolution of a cancer over time, during or subsequent to one or more treatment inventions (whether surgical or by radio, drug or immune therapy).

The term “purity” (also sometimes referred to as “tumour purity” or “tumour fraction” or ‘sample cellularity’ or aberrant cell fraction (ACF)) refers to the proportion of DNA containing cells within a mixed sample that are tumour cells, or to the equivalent proportion that is assumed to result in a particular mixture of genetic material from tumour and non-tumour cells in a sample. Several methods for determining the purity in a sample are known in the art. For example, in the context of cell or tissue samples, purity may be estimated by analysing pathology slides (e.g. hematoxylin and eosin (H&E)-stained slides or other histochemistry or immunohistochemistry slides, by counting tumour cells in one or more representative areas of a sample), or using high throughput assays such as flow cytometry. In the context of samples comprising genetic material, purity has been measured using sequence analysis processes that attempt to deconvolute tumour and germline genomes such as e.g. ASCAT (Van Loo et al., 2010), ABSOLUTE (Carter et al., 2012), or ichorCNA (Adalsteinsson et al., 2017). Advantageously, purity may be measured using the ECLIPSE method of the present invention, wherein one or more, preferably several, tumour-specific mutations are identified as being present in all cells of the tumour, i.e. the mutations are truly clonal. Determination that the tumour mutations are clonal may be carried out using known tools, such as PyClone (Roth, A., Khattra, J., Yap, D. et al. PyClone: statistical inference of clonal population structure in cancer. Nat Methods 11, 396-398 (2014). https://doi.org/10.1038/nmeth.2883) or DPclust (Nik-Zainal, Serena et al. “The life history of 21 breast cancers.” Cell vol. 149, 5 (2012): 994-1007. doi:10.1016/j.cell.2012.04.023.). As describe herein, when the mutations are known to be clonal, i.e. CCF=1, Equation 1A (see infra) may be rearranged to give Equation 2 (see infra), which calculates purity from CNₙₒᵣₘₐₗ, multiplicity, VAF and CNₜᵤₘₒᵤᵣ of a given clonal mutation. By combining (e.g. averaging) two or more mutation-specific purity values as given by Equation 2, it is possible to derive a reliable measure of sample purity (e.g. of a cfDNA-containing sample) even where purity is <5%.

A “normal sample” or “germline sample” refers to a sample that is assumed not to comprise tumour cells or genetic material derived from tumour cells. A germline sample may be a blood sample, a tissue sample, or a purified sample such as a sample of peripheral blood mononuclear cells from a subject. Similarly, the terms “normal”, “germline” or “wild type” when referring to sequences or genotypes refer to the sequence/genotype of cells other than tumour cells. A germline sample may comprise a small proportion of tumour cells or genetic material derived therefrom, and may nevertheless be assumed, for practical purposes, not to comprise said cells or genetic material. In other words, all cells or genetic material may be assumed to be normal and/or sequence data that is not compatible with the assumption may be ignored.

The term “sequence data” refers to information that is indicative of the presence and preferably also the amount of genomic material in a sample that has a particular sequence. Such information may be obtained using sequencing technologies, such as e.g. next generation sequencing (NGS), for example whole exome sequencing (WES), whole genome sequencing (WGS), or sequencing of captured genomic loci (targeted or panel sequencing), or using array technologies, such as e.g. copy number variation arrays, or other molecular counting assays. When NGS technologies are used, the sequence data may comprise a count of the number of sequencing reads that have a particular sequence. When non-digital technologies are used such as array technology, the sequence data may comprise a signal (e.g. an intensity value) that is indicative of the number of sequences in the sample that have a particular sequence, for example by comparison to an appropriate control. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular genomic location (where the “genomic location” refers to a location in the reference genome to which the sequence data was mapped). Further, a genomic location may contain a mutation, in which case counts of sequencing reads or equivalent non-digital signals may be associated with each of the possible variants (also referred to as “alleles”) at the particular genomic location. The process of identifying the presence of a mutation at a particular location in a sample is referred to as “variant calling” and can be performed using methods known in the art (such as e.g. the GATK HaplotypeCaller, https://gatk.broadinstitute.org/hc/en-us/articles/360037225632-HaplotypeCaller). For example, sequence data may comprise a count of the number of reads (or an equivalent non-digital signal) which match a germline (also sometimes referred to as “reference”) allele at a particular genomic location, and a count of the number of reads (or an equivalent non-digital signal) which match a mutated (also sometimes referred to as “alternate”) allele at the genomic location.

Further, sequence data may be used to infer copy number profiles along a genome, using methods known in the art. Copy number profiles may be allele specific. In the context of the present invention, copy number profiles are preferably allele specific and tumour/normal sample specific. In other words, the copy number profiles used in the present invention are preferably obtained using methods designed to analyse samples comprising a mixture of tumour and normal cells, and to produce allele-specific copy number profiles for the tumour cells and the normal cells in a sample. Allele specific copy number profiles for mixed samples may be obtained from sequence data (e.g. using read counts as described above), using e.g. ASCAT (Van Loo et al., 2010). Other methods are known and equally suitable. Preferably, within the context of the present invention, the method used to obtain allele-specific copy number profiles is one that reports a plurality of possible copy number solutions and an associated quality/confidence metric. For example, ASCAT outputs a goodness-of-fit metric for each combination of values of ploidy (ploidy for a whole tumour sample, not segment-specific) and purity for which a corresponding allele-specific copy number profile was evaluated. Note that the tumour-specific copy number profiles generated by such methods represent an average or summary of the entire tumour cell population (i.e. it does not account for heterogeneity within the tumour population).

The term “total copy number” refers to the total number of copies of a genomic region in a sample. The term “major copy number” refers to the number of copies of the most prevalent allele in a sample. Conversely, the term “minor copy number” refers to the number of copies of the allele other than the most prevalent allele in a sample. Unless indicated otherwise, these terms refer to the inferred major and major copy numbers (and total copy numbers) for an inferred tumour copy number profile. The term “normal copy number” or “normal total copy number” refers to the number of copies of a genomic region in the normal cells in a sample.

Normal cells typically have two copies of each chromosome (unless the cell is genetically male and the chromosome is a sex chromosome), and hence the normal copy number may in embodiments be assumed to be equal to 2 (unless the genomic region is on the X or Y chromosome and the sample under analysis is from a male subject, in which case the normal copy number may be assumed to be equal to 1). Alternatively, the normal copy number for a particular genomic region may be determined using a normal sample.

The terms “tumour-specific mutation”, “somatic mutation” or simply “mutation” are used interchangeably and refer to a difference in a nucleotide sequence (e.g. DNA or RNA) in a tumour cell compared to a healthy cell from the same subject. The difference in the nucleotide sequence can result in the expression of a protein which is not expressed by a healthy cell from the same subject. For example, a mutation may be a single nucleotide variant (SNV), multiple nucleotide variant (MNV), a deletion mutation, an insertion mutation, a translocation, a missense mutation, a translocation, a fusion, a splice site mutation, or any other change in the genetic material of a tumour cell. A mutation may result in the expression of a protein or peptide that is not present in a healthy cell from the same subject. Mutations may be identified by exome sequencing, RNA-sequencing, whole genome sequencing and/or targeted gene panel sequencing and or routine Sanger sequencing of single genes, followed by sequence alignment and comparing the DNA and/or RNA sequence from a tumour sample to DNA and/or RNA from a reference sample or reference sequence (e.g. the germline DNA and/or RNA sequence, or a reference sequence from a database). Suitable methods are known in the art.

An “indel mutation” refers to an insertion and/or deletion of bases in a nucleotide sequence (e.g. DNA or RNA) of an organism. Typically, the indel mutation occurs in the DNA, preferably the genomic DNA, of an organism. An indel mutation may be a frameshift indel mutation. A frameshift indel mutation is a change in the reading frame of the nucleotide sequence caused by an insertion or deletion of one or more nucleotides. Such frameshift indel mutations may generate a novel open-reading frame which is typically highly distinct from the polypeptide encoded by the non-mutated DNA/RNA in a corresponding healthy cell in the subject.

A “neoantigen” (or “neo-antigen”) is an antigen that arises as a consequence of a mutation within a cancer cell. Thus, a neoantigen is not expressed (or expressed at a significantly lower level) by normal (i.e. non-tumour) cells. A neoantigen may be processed to generate distinct peptides which can be recognised by T cells when presented in the context of MHC molecules. Neoantigens may be used as the basis for cancer immunotherapies. References herein to “neoantigens” are intended to include also peptides derived from neoantigens. The term “neoantigen” as used herein is intended to encompass any part of a neoantigen that is immunogenic. An “antigenic” molecule as referred to herein is a molecule which itself, or a part thereof, is capable of stimulating an immune response, when presented to the immune system or immune cells in an appropriate manner. The binding of a neoantigen to a particular MHC molecule (encoded by a particular HLA allele) may be predicted using methods which are known in the art. Examples of methods for predicting MHC binding include those described by Lundegaard et al., O'Donnel et al., and Bullik-Sullivan et al. For example, MHC binding of neoantigens may be predicted using the netMHC-3 (Lundegaard et al.) and netMHCpan4 (Jurtz et al.) algorithms. A neoantigen that has been predicted to bind to a particular MHC molecule is thereby predicted to be presented by said MHC molecule on the cell surface.

A “clonal neoantigen” is a neoantigen that results from a mutation that is present in essentially every tumour cell in one or more samples from a subject (or that can be assumed to be present in essentially every tumour cell from which the tumour genetic material in the sample(s) is derived). Similarly, a “clonal mutation” is a mutation that is present in essentially every tumour cell in one or more samples from a subject (or that can be assumed to be present in essentially every tumour cell from which the tumour genetic material in the sample(s) is derived). Thus, a clonal mutation may be a mutation that is present in every tumour cell in one or more samples from a subject. A “sub-clonal” neoantigen is a neoantigen that results from a mutation that is present in a subset or a proportion of cells in one or more tumour samples from a subject (or that can be assumed to be present in a subset of the tumour cells from which the tumour genetic material in the sample(s) is derived). Similarly, a “sub-clonal” mutation is a mutation that is present in a subset or a proportion of cells in one or more tumour samples from a subject (or that can be assumed to be present in a subset of the tumour cells from which the tumour genetic material in the sample(s) is derived). As the skilled person understands, a neoantigen or mutation may be clonal in the context of one or more samples from a subject while not being truly clonal in the context of the entirety of the population of tumour cells that may be present in a subject (e.g. including all regions of a primary tumour and metastasis). Thus, a clonal mutation may be “truly clonal” in the sense that it is a mutation that is present in essentially every tumour cell (i.e. in all tumour cells) in the subject. This is because the one or more samples may not be representative of each and every subset of cells present in the subject.

The term “cancer cell fraction” (or “CCF”) refers to the proportion of tumour cells that contain a mutation. Within the context of the present invention, the cancer cell fraction may be estimated based on one or more samples, and as such may not be equal to the true cancer cell fraction in the subject. Without wishing to be bound by any particular theory, the present inventors believe that the ECLIPSE method of the invention described herein is, in some embodiments, able to provide a more representative and therefore more accurate CCF for a given mutation or given sub-clonal population of tumour cells than is seen with CCF estimates based on tissue sampling alone. This is because sampling of cfDNA-containing samples (such as plasma samples) tends to minimise sampling bias and captures, in principle, ctDNA shed by all cells making up one or more tumours of the patient. Nevertheless, the cancer cell fraction estimated based on one or more samples may provide a useful indication of the likely true cancer cell fraction.

A cancer immunotherapy (or simply “immunotherapy”) refers to a therapeutic approach comprising administration of an immunogenic composition (e.g. a vaccine), a composition comprising immune cells, or an immunoactive drug, such as e.g. a therapeutic antibody, to a subject. The term “immunotherapy” may also refer to the therapeutic compositions themselves. In the context of the present invention, the immunotherapy typically targets a neoantigen. For example, an immunogenic composition or vaccine may comprise a neoantigen, neoantigen presenting cell or material necessary for the expression of the neoantigen. As another example, a composition comprising immune cells may comprise T and/or B cells that recognise a neoantigen. The immune cells may be isolated from tumours or other tissues (including but not limited to lymph node, blood or ascites), expanded ex vivo or in vitro and re-administered to a subject (a process referred to as “adoptive cell therapy”). Instead or in addition to this, T cells can be isolated from a subject and engineered to target a neoantigen (e.g. by insertion of a chimeric antigen receptor that binds to the neoantigen) and re-administered to the subject. As another example, a therapeutic antibody may be an antibody which recognises a neoantigen.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

References to “an immune cell” are intended to encompass cells of the immune system, for example T cells, NK cells, NKT cells, B cells and dendritic cells. In a preferred embodiment, the immune cell is a T cell. An immune cell that recognises a neoantigen may be an engineered T cell. A neoantigen specific T cell may express a chimeric antigen receptor (CAR) or a T cell receptor (TCR) which specifically binds a neoantigen or a neoantigen peptide, or an affinity-enhanced T cell receptor (TCR) which specifically binds a neoantigen or a neoantigen peptide (as discussed further hereinbelow). For example, the T cell may express a chimeric antigen receptor (CAR) or a T cell receptor (TCR) which specifically binds to a neo-antigen or a neo-antigen peptide (for example an affinity enhanced T cell receptor (TCR) which specifically binds to a neo-antigen or a neo-antigen peptide). Alternatively, a population of immune cells that recognise a neoantigen may be a population of T cell isolated from a subject with a tumour. For example, the T cell population may be generated from T cells in a sample isolated from the subject, such as e.g. a tumour sample, a peripheral blood sample or a sample from other tissues of the subject. The T cell population may be generated from a sample from the tumour in which the neoantigen is identified. In other words, the T cell population may be isolated from a sample derived from the tumour of a patient to be treated, where the neoantigen was also identified from a sample from said tumour. The T cell population may comprise tumour infiltrating lymphocytes (TIL).

The term “Antibody” (Ab) includes monoclonal antibodies, polyclonal antibodies, multispecific antibodies (e.g., bispecific antibodies), and antibody fragments that exhibit the desired biological activity. The term “immunoglobulin” (lg) may be used interchangeably with “antibody”. Once a suitable neoantigen has been identified, for example by a method according to the invention, methods known in the art can be used to generate an antibody.

An “immunogenic composition” is a composition that is capable of inducing an immune response in a subject. The term is used interchangeably with the term “vaccine”. The immunogenic composition or vaccine described herein may lead to generation of an immune response in the subject. An “immune response” which may be generated may be humoral and/or cell-mediated immunity, for example the stimulation of antibody production, or the stimulation of cytotoxic or killer cells, which may recognise and destroy (or otherwise eliminate) cells expressing antigens corresponding to the antigens in the vaccine on their surface.

As used herein “treatment” refers to reducing, alleviating or eliminating one or more symptoms of the disease which is being treated, relative to the symptoms prior to treatment. “Prevention” (or prophylaxis) refers to delaying or preventing the onset of the symptoms of the disease. Prevention may be absolute (such that no disease occurs) or may be effective only in some individuals or for a limited amount of time.

As used herein, the terms “computer system” includes the hardware, software and data storage devices for embodying a system or carrying out a method according to the above described embodiments. For example, a computer system may comprise a central processing unit (CPU), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. It is explicitly envisaged that computer system may consist of or comprise a cloud computer.

As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Applications

As shown in Examples 2 and 3 herein, the ECLIPSE method of the present invention provides a reliable and in many cases improved estimate of CCF from a typically more easily obtained sample (e.g. a plasma sample). Further features of the present method, such as the ability to determine, with statistical confidence, whether a cancer-specific mutation that is or was present in the tumour tissue sample, is absent from the plasma sample facilitate the monitoring of clonal dynamics of a tumour. The present invention therefore finds considerable application in the field of cancer treatment, cancer management, diagnostics and prognostics. In some embodiments, the at least one tumour-specific mutation results in the mutated DNA encoding a neoantigen and/or the at least one tumour-specific mutation is or encodes a target for an anti-cancer therapy. A neoantigen that is clonal or predicted to become clonal will typically be a more attractive target for, e.g., a cancer vaccine, a T cell therapy, a CAT-T therapy or other cell therapy. In some cases, a neoantigen found to be present only a in branch/subclonal population may be a suitable target for vaccine, T cell therapy, CAR-T, etc., because the mutation giving rise to the neoantigen is growing and/or approaching clonality. On the other hand, the method of the present invention may be used to detect that a formerly clonal mutation has a CCF<1, and is no longer predicted to be a good target.

As the skilled person will appreciate, a number of modern anti-cancer therapeutics have been approved for, or a more efficacious when, the subject being treated has a cancer harbouring one or more specific mutations. Examples of such “targeted cancer therapy” are described in Baudino T A. Targeted Cancer Therapy: The Next Generation of Cancer Treatment. Curr Drug Discov Technol. 2015; 12 (1): 3-20. doi: 10.2174/1570163812666150602144310. PMID: 26033233, which is incorporated herein by reference. The ECLIPSE method of the present invention may find use in the context of targeted cancer therapy. In particular, when the one or more cancer-specific mutations represent targets for such targeted therapy, information about the CCF of the cancer cells harbouring those mutations provides a valuable treatment insight. For example, knowledge that a mutation that is a therapeutic target is clonal or is approaching clonality may make the corresponding therapy more attractive for this subject. On the other hand, a high CCF of a mutation that confers resistance to an anti-cancer therapy may make that therapy less attractive and/or result in a worse prognosis for that subject.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Example 1—Illustrative Eclipse Worked Example

To accurately estimate CCF of mutations, and thus accurately estimate the clonal architecture and its dynamics overtime time in low purity ctDNA samples, the present inventors have developed a method which takes estimates of the copy number state at each mutated position and the clonal groups to which each mutation belongs from matched tumour tissue sequencing and uses this information with a reconfiguration of the equations from tissue clonality extraction methods and estimates of background sequencing noise to accurately deconvolve the clonal architecture. More precisely, CCFs can be estimated suing the following equation:

$$multiplicity * ccf = VAF \frac{1}{Purity} ( Purity \times {CN}_{tumour} + ( 1 - Purity ) \times {CN}_{normal} ) \qquad ( Equation 1 A )$$

Equation 1A can be rearranged for CCF, as follows:

$$CCF = \qquad ( Equation 1 B )$$

$$VAF \frac{1}{multiplicity * Purity} ( Purity \times {CN}_{tumour} + ( 1 - Purity ) \times {CN}_{normal} )$$

VAF (variant allele fraction) of mutations can be measured from the ctDNA data using targeted deep sequencing. A mutation's multiplicity, the tumour copy number and the normal copy number at the mutated site can be estimated from whole exome sequencing of tumour tissue from any timepoint. It is also possible to determine which mutations are clonal using the tumour tissue and additional filters from the VAF distribution in the ctDNA. Hence for these clonal mutations (where CCF=1) the purity of the sample can be estimated by rearranging this equation:

$$Purity = \frac{{CN}_{norm}}{\frac{mulitplicity}{VAF} - {CN}_{tum} + {CN}_{norm}} \qquad ( Equation 2 )$$

VAFs can also be corrected using provided background noise estimates and all factors are then known for the first equation (i.e. Equation 1A/1B) to identify CCFs for each mutation. A number of additional filters can then be applied using the distribution of these CCFs, which known tumour clones from tissue sequencing to identify outliers where either the clone identity or copy number information from a given mutation, estimated from the tumour tissue, may be incorrect in this ctDNA sample.

Extraction of Clonality from Liquid bioPSIEs (ECLIPSE) may also perform a number of other estimations including a statistical test to estimate the presence or absence or each subclone in each sample using the provided background noise estimates and a test for whether each subclone is clonal (in 100% of cells) or subclonal (in a minority of cells). In some cases the statistical test may be selected from the group consisting of: a binomial test, a Poisson test, a one sample Wilcoxon rank sum test (using an expected background distribution), a chi-squared test and a Fisher's exact test (comparing the expected reference and variant counts to the observed reference and variant counts).

Table 1 shows 9 columns (1-9) of representative data input into the ECLIPSE tool (provided as an R package).

TABLE 1
data inputs for ECLIPSE

| | 2. | | 4. | 5. | 6. | | 8. | 9. |
|---|---|---|---|---|---|---|---|---|
| 1. | variant_ | 3. | background_ | is_ | clone_ | 7. | tumour_ | normal_ |
| mutation_id | reads | depth | error_rate | clonal | name | multiplicity | total_cpn | total_cpn |
| mutation 1 | 180 | 3610 | 0.0000455 | TRUE | 1 | 2.367957535 | 3.421333333 | 2 |
| 10:28445525:T | | | | | | | | |
| mutation 2 | 96 | 3600 | 0.00000929 | TRUE | 1 | 1.980712748 | 2 | 2 |
| 12:11851027:G | | | | | | | | |
| mutation 3 | 67 | 3529 | 0.0000332 | TRUE | 1 | 1.906209094 | 2 | 2 |
| 18:64735686:C | | | | | | | | |
| mutation 4 | 131 | 4980 | 0.00000504 | TRUE | 1 | 1.38184391 | 2.578666667 | 2 |
| 11:12989067:A | | | | | | | | |
| mutation 5 | 143 | 4015 | 0.0000159 | TRUE | 1 | 3.624994881 | 5.288 | 2 |
| 17:67061519:T | | | | | | | | |
| mutation 6 | 90 | 3364 | 0.00000585 | TRUE | 1 | 2.130989773 | 3.394666667 | 2 |
| 8:68638557:G | | | | | | | | |
| mutation 7 | 93 | 3031 | 0.0000105 | TRUE | 1 | 2.525062772 | 4.237333333 | 2 |
| 16:61285347:G | | | | | | | | |
| mutation 8 | 114 | 3351 | 0.00000879 | TRUE | 1 | 2.013661511 | 4.421333333 | 2 |
| 15:54449010:A | | | | | | | | |
| mutation 9 | 127 | 2484 | 0.0000131 | TRUE | 1 | 2.488812176 | 2.205333333 | 2 |
| 1:37736112:C | | | | | | | | |
| mutation 10 | 264 | 4342 | 0.0001262 | TRUE | 1 | 2.537105919 | 2.205333333 | 2 |
| 5:9640844:C | | | | | | | | |
| mutation 11 | 110 | 3259 | 0.0000106 | TRUE | 1 | 1.707742024 | 4.290666667 | 2 |
| 21:52863373:T | | | | | | | | |
| mutation 12 | 195 | 3904 | 0.00000917 | TRUE | 1 | 2.188259044 | 4.712 | 2 |
| 4:36153694:T | | | | | | | | |
| mutation 13 | 187 | 3711 | 0.0000443 | TRUE | 1 | 2.06483407 | 4.712 | 2 |
| 9:7271997:T | | | | | | | | |
| mutation 14 | 166 | 3202 | 0.0000297 | TRUE | 1 | 2.162372062 | 4.506666667 | 2 |
| 15:33861954:T | | | | | | | | |
| mutation 15 | 52 | 2879 | 0.00000221 | TRUE | 1 | 2.949598256 | 5.317333333 | 2 |
| 9:51839167:G | | | | | | | | |
| mutation 16 | 5 | 4447 | 0.00000929 | FALSE | 3 | 1 | 2.845333333 | 2 |
| 14:91892997:T | | | | | | | | |
| mutation 17 | 28 | 3976 | 0.000157768 | FALSE | 3 | 1.707657055 | 3.394666667 | 2 |
| 2:91153755:C | | | | | | | | |
| mutation 18 | 8 | 2771 | 0.0000144 | FALSE | 3 | 1.086214286 | 4.237333333 | 2 |
| 21:38300742:G | | | | | | | | |
| mutation 19 | 8 | 2166 | 0.0000207 | FALSE | 3 | 1 | 4.205333333 | 2 |
| 9:13911502:G | | | | | | | | |
| mutation 20 | 7 | 3598 | 0.0000339 | FALSE | 3 | 1 | 3.776 | 2 |
| 20:23468462:A | | | | | | | | |
| mutation 21 | 11 | 3593 | 0.0000144 | FALSE | 3 | 1.581214286 | 4.832 | 2 |
| 2:88899388:A | | | | | | | | |
| mutation 22 | 0 | 2591 | 0.0000183 | FALSE | 8 | 1.168421053 | 2.389333333 | 2 |
| 19:59660795:A | | | | | | | | |
| mutation 23 | 0 | 3191 | 0.00000848 | FALSE | 8 | 1 | 3.794666667 | 2 |
| 15:34506483:C | | | | | | | | |
| mutation 24 | 1 | 1180 | 0.0000248 | FALSE | 8 | 1 | 3.794666667 | 2 |
| 18:66853871:T | | | | | | | | |
| mutation 25 | 0 | 2054 | 0.00000673 | FALSE | 8 | 1 | 3.794666667 | 2 |
| 17:25931170:G | | | | | | | | |
| mutation 26 | 0 | 3685 | 0.0000447 | FALSE | 8 | 1 | 3.626666667 | 2 |
| 7:89606347:A | | | | | | | | |
| mutation 27 | 1 | 3288 | 0.000157768 | FALSE | 8 | 1 | 4.624 | 2 |
| 10:96171281:C | | | | | | | | |
| mutation 28 | 0 | 2405 | 0.000122706 | FALSE | 8 | 1.04040404 | 3.832 | 2 |
| 19:26432411:C | | | | | | | | |

Column 1 is the id for each mutation. Also shown are the chromosome, position and alternate base (e.g. 10:28445525:T indicates chromosome: 10, position: 28445525, base: T).
Column 2 is the number of mutated reads observed for each mutation in plasma.
Column 3 is the depth sequenced for each mutated position in plasma.
Column 4 is the background error rate for each mutation in the plasma (i.e. the probability that each read will contain this mutation by chance).
Column 5 indicates whether the mutation is clonal (in every tumour cell) or not (measured from tissue samples). The determination of whether a mutation is or is not clonal is described below.
Column 6 indicates which clone each mutation belongs to (measured from tissue samples). The determination of which clone each mutation belongs to may be performed as described in detail below.
Column 7 indicates the multiplicity of the mutation (number of DNA copies of the mutation, measured from tissue samples).
Column 8 indicates the total copy number in tumour cells (number of wildtype and mutant copies) at the mutated locus (measured from tissue samples).
Column 9 indicates the total copy number in non-tumour cells (this is usually presumed to be 2 as normal cells are diploid, but could be measured from tissue samples).

### Background Error Rate Calculation

For this example, background error rate was calculated from non-mutated genome positions that were sequenced in each plasma sample. Our sequencing of plasma targets known mutations identified in tumour tissue exome sequencing. However, we also sequenced several hundred base pairs up and downstream of these known mutations. We refer to all positions sequenced in plasma which were not already known to be mutated from the tissue exome sequencing to be non-mutated positions. It is, however, specifically contemplated herein that the background error may be calculated using other methods as known in the literature which could be used and inputted into ECLIPSE.

Determination of which Mutations are in which Clone and Whether a Mutation is or is not Clonal

There are several methodologies for determining which mutations belong to what clones in sequencing data of tumour tissue. These all rely on clustering those mutations based on their estimated CCFs. CCFs were calculated using Equation 1B as shown above, where the copy number and purity estimates come from application of a tool such as ASCAT (github.com/VanLoo-lab/ascat) which leverages coverage of germline single nucleotide polymorphisms in the exome/whole genome. In the present example, PyClone was used to perform this. A guide to the PyClone tool can be found at the following site: github.com/Roth-Lab/pyclone (incorporated herein by reference). Alternatives to PyClone which also provide the required information are include Decifer described at github.com/raphael-group/decifer (incorporated herein by reference) and DPclust, described at github.com/Wedge-lab/dpclust (incorporated herein by reference).

Once the clone identity is confirmed using PyClone or similar method (such as Decifer or DPclust) the determination of which of these clones is the clonal cluster (and hence the mutations assigned to it are clonal mutations) can be done by calculating the mean of the CCF (cancer cell fraction) for all mutations in each clone and assigned the clone with the highest mean CCF to be clonal.

Table 2 shows 7 columns (10-16) calculated from the input data of Table 1 by the ECLIPSE tool:

TABLE 2
calculated values from Table 1 data by ECLIPSE

| | 11. | | 13. | | | 16. |
|---|---|---|---|---|---|---|
| 10. | clone_present | 12. | purity_ | 14. | 15. | clone_ |
| error_reads | p-value | vaf | estimate | purity | ccf | ccf |
| 0.164255 | 0 | 0.049861496 | 0.043412792 | 0.035 | 1.22157475 | 0.996 |
| 0.033444 | 0 | 0.026666667 | 0.026926334 | 0.035 | 0.761909051 | 0.996 |
| 0.1171628 | 0 | 0.018985548 | 0.019919691 | 0.035 | 0.563648693 | 0.996 |
| 0.0250992 | 0 | 0.026305221 | 0.038496705 | 0.035 | 1.088321144 | 0.996 |
| 0.0638385 | 0 | 0.035616438 | 0.020306485 | 0.035 | 0.588336361 | 0.996 |
| 0.0196794 | 0 | 0.026753864 | 0.025556821 | 0.035 | 0.72800462 | 0.996 |
| 0.0318255 | 0 | 0.030682943 | 0.024981891 | 0.035 | 0.714857672 | 0.996 |
| 0.02945529 | 0 | 0.034019696 | 0.035230051 | 0.035 | 0.996999453 | 0.996 |
| 0.0325404 | 0 | 0.051127214 | 0.041259673 | 0.035 | 1.166779538 | 0.996 |
| 0.5479604 | 0 | 0.060801474 | 0.048166806 | 0.035 | 1.361144732 | 0.996 |
| 0.0345454 | 0 | 0.033752685 | 0.04140352 | 0.035 | 1.163789275 | 0.996 |
| 0.03579968 | 0 | 0.04994877 | 0.048664088 | 0.035 | 1.35366398 | 0.996 |
| 0.1643973 | 0 | 0.05039073 | 0.052267814 | 0.035 | 1.447272458 | 0.996 |
| 0.0950994 | 0 | 0.051842598 | 0.051015636 | 0.035 | 1.416885775 | 0.996 |
| 0.00636259 | 0 | 0.018061827 | 0.012500913 | 0.035 | 0.366854715 | 0.996 |
| 0.04131263 | 0 | 0.001124353 | | 0.035 | 0.064579992 | 0.151 |
| 0.627285568 | 0 | 0.007042254 | | 0.035 | 0.239133256 | 0.151 |
| 0.0399024 | 0 | 0.002887044 | | 0.035 | 0.156362534 | 0.151 |
| 0.0448362 | 0 | 0.003693444 | | 0.035 | 0.217165077 | 0.151 |
| 0.1219722 | 0 | 0.001945525 | | 0.035 | 0.113556637 | 0.151 |
| 0.0517392 | 0 | 0.003061508 | | 0.035 | 0.115055513 | 0.151 |
| 0.0474153 | 0.2995538 | 0 | | 0.035 | 0 | 0.01 |
| 0.02705968 | 0.2995538 | 0 | | 0.035 | 0 | 0.01 |
| 0.029264 | 0.2995538 | 0.000847458 | | 0.035 | 0.04948032 | 0.01 |
| 0.01382342 | 0.2995538 | 0 | | 0.035 | 0 | 0.01 |
| 0.1647195 | 0.2995538 | 0 | | 0.035 | 0 | 0.01 |
| 0.518741184 | 0.2995538 | 0.000304136 | | 0.035 | 0.018009766 | 0.01 |
| 0.29510793 | 0.2995538 | 0 | | 0.035 | 0 | 0.01 |

Column 10 is the number of mutated reads we would expect to observe by chance due to background error for each mutation. This is calculated by multiplying the entry in column 3 of Table 1 by the entry in column 4 of Table 1.
Column 11 is a P value for each clone indicating the probability of obtaining the observed result when the null hypothesis is actually true, e.g., a low P-value (P<0.01) means that it is very unlikely that the we would find the observed number of variant reads for the clone if the clone is, in fact, not present. The ECLIPSE method may provide a read-out of the P-value allowing the user to set an appropriate threshold. For example, a clone may be considered to be not present when the P-value is greater than 0.05 or when the P-value is greater than 0.01. As the skilled person will appreciate, the chosen P-value threshold should be tailored to the analysis being performed depending on the number of tests and the acceptable Type I/II error overall.
   This is calculated by summing column 2 (in Table 1) to give the total number of reads observed across all the mutations in each clone (for example, 2015 variant reads for clone 1), summing column 10 (in Table 2) to give the total number of reads we would expect by chance across all the mutations in each clone, and then applying a statistical test where the sum of column 10 is the background lambda and sum of column 2 is our observation for each clone to give a P value for whether we have more signal than the estimated noise, hence whether the clone is present. In particular, the statistical test may be a binomial test, a Poisson test, a one sample Wilcoxon rank sum test, or a chi-squared/Fisher's exact test.
Column 12 is the variant allele fraction (VAF) for each mutation in the plasma, which is determined by dividing the entries in column 2 of Table 1 by those of column 3 of Table 1.
Column 13 indicates mutation-specific estimates of the purity. The mutation-specific purity estimates are derived from the values of CNₙₒᵣₘ (column 9 in Table 1), multiplicity (column 7 in Table 1), VAF (column 12 in Table 2) and CNₜᵤₘ (column 8 in Table 1) which are inputted in equation 2 for mutations of a clone known to be clonal from prior tissue sampling (i.e. where “is clonal?” is “TRUE” in column 5 of Table 1. The output of equation 2 is the mutation-specific estimate of purity.
Column 14 is the mean of column 13 of Table 2 across all clonal mutations in a sample—i.e. the final estimate of the purity for a plasma sample.
Column 15 is the Cancer Cell fraction (CCF) estimate for each mutation by applying Equation 1B. That is, for each mutation, VAF is taken from column 12 of Table 2, multiplicity is taken from column 7 of Table 1, purity (P) is taken from column 14 of Table 2 (i.e. the final estimate of purity for the plasma sample), CNₜᵤₘ is taken from column 8 of Table 1, and CNₙₒᵣₘ is taken from column 9 of Table 1.
Column 16 shows the Clone-level estimates of CCF calculated by averaging the CCF across all mutations in a given clone.

In this Example, we see that—as expected for a clone having “is clonal=true”—the estimated CCF of clone 1 is very close to 1 (0.996). The estimated CCF of clone 3 is 0.151, i.e. around 15%. The estimated CCF of clone 8 is close to zero (0.01), which is expected since the P-value of around 0.3 indicates that the clone is unlikely to be present (because there is a high probability that the number of observed variant reads is attributable to chance alone in view of the background sequencing error rate).

These results provide potentially actionable clinical intelligence because we are able to conclude that the sample has a tumour purity of 3.5% and subclone 3 was present (p value is very low for column 11) and in ˜15% of tumour cells (column 16) at the time when this plasma sample was taken. This would indicate that the mutations in subclone 3 were poor targets at this time point as they are not present in most cells, but this may change over time. If later plasma samples are processed through the ECLIPSE pipeline of the present invention, it may be found that subclone 3 becomes truly clonal and then the mutations in it would be targetable. In the present Example, we did not see any evidence that subclone 8 is present in this sample (column 11—P value is too high). This means that it is unlikely that subclone 8 will become clonal (and its mutations targetable) in the future.

### Example 2—Eclipse Validation

Background: Outgrowth of resistance cancer cell populations is a common mechanism of therapy failure in oncology. Effective personalised medicine is reliant on targeting aberrations that are present in every tumour cell, however tumours are heterogenous and comprehensive tumour tissue sampling is often impossible. Liquid biopsies have the potential to provide representative tumour sampling at regular intervals through disease course, but current clonal deconvolution methods are ineffective in low tumour content samples (<5%) which comprise most samples in the localised or minimal residual disease (MRD) settings.

Methods: The present inventors analysed 1092 plasma samples from 201 patients enrolled in the TRACERx study of early stage non-small cell lung cancer (NSCLC) who also underwent multiregional whole exome sequencing (WES) of primary tumour and relapse tissue. Personalised panels were designed targeting 200 mutations and sequenced plasma to a median unique depth of 2149X. The informatic tool ECLIPSE (Extraction of CLonality from Liquid bioPSiEs) was designed to leverage variant allele fractions (VAFs) and background noise estimates from plasma, with copy number and clone identities per mutation called from tumour tissue to accurately estimate plasma sample purity, the presence or absence of subclones and their cancer cell fraction (CCF) at the time of plasma sample collection. Using simulations, the present inventors estimated that ECLIPSE was powered to detect 10% CCF subclones at purities of 0.2%. Only samples with >0.2% purity (52% of MRD positive samples) were considered for clonality analysis.

Results: To validate the use of liquid biopsies and ECLIPSE for representative sampling of intra-tumour heterogeneity the present inventors compared clonal deconvolution using ECLIPSE in plasma samples collected before surgery to those estimated via multiregional exome sequencing at surgery. The present inventors found a 1:1 correlation between CCFs of subclones estimated in plasma and tissue in these samples (P<0.001, R²=0.6, mean purity=1.4%) whereas VAF-only estimates of CCF in plasma systematically underestimated CCFs when compared to tissue samples, misclassifying potential therapeutic targets. ECLIPSE detected 97% of subclones present in multiple regions across the tumour tissue and 63% of subclones which were unique to a single region. Analysing the CCFs estimates in single region unique subclones the present inventors found CCFs in plasma were consistently lower than those found in tissue (P<0.001, OR=0.33), particularly in larger tumours where a smaller proportion of tumour tissue was sampled (P<0.001, OR=0.16). This effect was not apparent in subclones spread across several tumour regions. This is consistent with sampling bias due to spatial constraints in the primary tumour, which is overcome using plasma sequencing. Illusion of clonality, where variants are ubiquitous in a sample but not in the unsampled tumour, is common in single tissue region sampling. The present inventors found significantly lower plasma CCFs in clonal illusion mutations from randomly selected regions in each TRACERx patient when compared to true clonal mutations, distinguishing appropriate therapeutic targets, such as neoantigens, without a requirement for multiregional sampling. The clonal structure at metastasis detected in 28 patients was compared with both tissue and cfDNA sampling at relapse. The present inventors detected 28/29 of the subclones found in the relapsing tissue in the corresponding cfDNA. Of the 125 subclones tracked in the cfDNA from the primary tumour that were absent in relapsing tissue the present inventors found an additional 8 subclones from 7 patients present in cfDNA at relapse. A strong bias was found for these 8 subclones to be subclonal at the point of relapse in the cfDNA (P=0.008, OR=5.5). In addition, a trend was found towards a higher number of unsampled metastatic sites in these 7 patients (P=0.19) consistent with these subclones being missed in tissue due to insufficient sampling. In patients with polyclonal relapses, the present inventors observed clonal dynamics over time, some of which were concurrent with treatment. Finally, the present inventors found that in relapsing patients, metastatic competent subclones have a higher CCF in the primary tumour, i.e. a larger clone size, as measured in pre-operative plasma, than clones which do not metastasise (P<0.001, OR=4.5) and that relapsing patients in general have larger subclones in their primary tumours than non-relapsing patients (P=0.043).

Conclusions: The present inventors have found evidence that plasma sampling can accurately profile the clonal structure of tumours over time using the informatic tool ECLIPSE, in accordance with the present invention, revealing biological determinants of metastasis, clonal dynamics in response to treatment and with the potential to better tailor targeted therapeutics to variants which are present in all tumour cells.

### Example 3—Clonal Deconvolution of Plasma Samples Using Eclipse

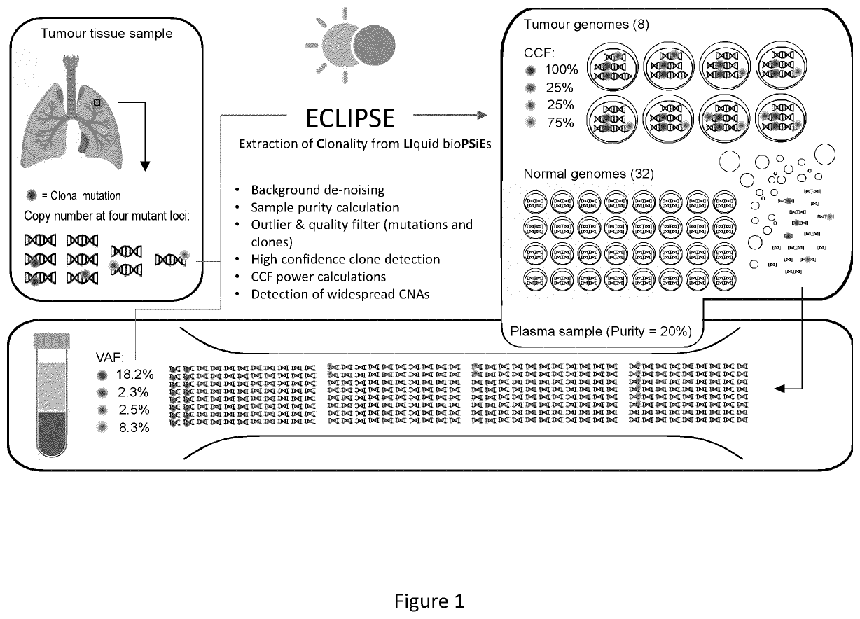

The informatic tool ECLIPSE (Extraction of CLonality from Liquid bioPSiEs) was developed to overcome the challenges of performing clonal deconvolution in ultra-low cellularity plasma samples. ECLIPSE employs measures of mutation variant allele fractions (VAFs) from plasma which for ultra-low purity samples can be assessed with several deep targeted sequencing methods in combination with data on the clonal status of each mutation, and its copy number state from a tumour tissue sample. Referring to FIG. 1, on the right are shown four example mutations, each belonging to a separate clone. In this example, a tumour with 8 cells. The blue cells are clonal and have two mutant copies, where are the other three mutations are in different subsets of the tumour cells with different copy number statuses. As expected, we see that the cancer cell fraction (CCF) and the number of mutated and wildtype copes has an effect on the variant allele fractions (VAFs) when these DNA molecules are extruded into the plasma as shown at the bottom. We observe these VAFs along with the copy number and clonal status of these different mutations as shown in the top left from the tumour tissue, in this case collected at baseline, and we then use the clonal mutations to calculate the purity of the sample from which we can determine CCFs for mutations and clones in the plasma samples over time.

The following steps outline the method for obtaining the necessary inputs for the ECLIPSE tool, which then outputs an estimate of the CCF for a given (e.g. sub-clonal) clone:
1) Collect at least 1 tumour sample;
2) DNA extraction and whole exome sequencing (WES) (or whole genome sequencing (WGS)) from the tumour sample;
3) Run mutation and copy number calling (e.g. using Mutect and ASCAT) and use the output from these methods to run a clonal deconvolution tool such as PyClone (or Dpclust or others) that outputs which mutations belong to which clones;
4) Use these outputs to calculate which mutations are clonal and the copy number at each mutated locus;
5) Collect one or more ctDNA-containing samples (e.g. a plasma samples) at one or more time points for this patient;
6) Perform an assay to calculate variant allele fractions (VAFs) for every mutation of interest (e.g. potential neoantigens or targetable mutations such as those conferring sensitivity to tyrosine kinase inhibitors) from the tissue sequencing in each plasma sample. This could be WES (in particular, for late stage patients with high tumour and ctDNA burden) but may preferably be an error-corrected targeted deep sequencing method like the ArcherDx MRD method (for example, in the context of earlier stage patients across a wider variety of cancer types);
7) Estimate background sequencing noise for each mutation followed in plasma by looking at non-mutated positions in the genome (see details provided in Example 1 under the heading “Background error rate calculation”);
8) Input the mutation and total copy numbers, background sequencing noise, the clone membership of each mutation and the plasma variant allele fractions into the ECLIPSE informatic tool (see Example 1).

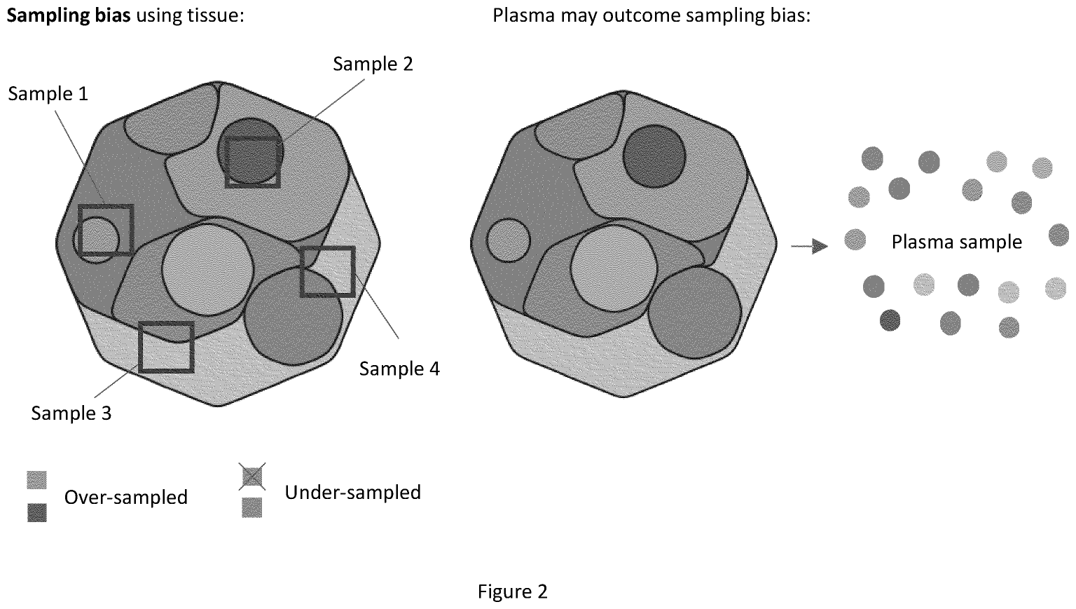

As shown in FIG. 2, plasma sampling has the potential to capture more of the heterogeneity of the tumour than even multi-regional tissue sampling. In particular, tissue sampling can result in significant sampling bias due the fact that spatially restricted subclones are likely to be either missed (e.g. purple) or overestimated (orange and brown). On the other hand all, or at least most, cells in the tumour will shed ctDNA into the plasma, hence allowing more representative sampling through time.

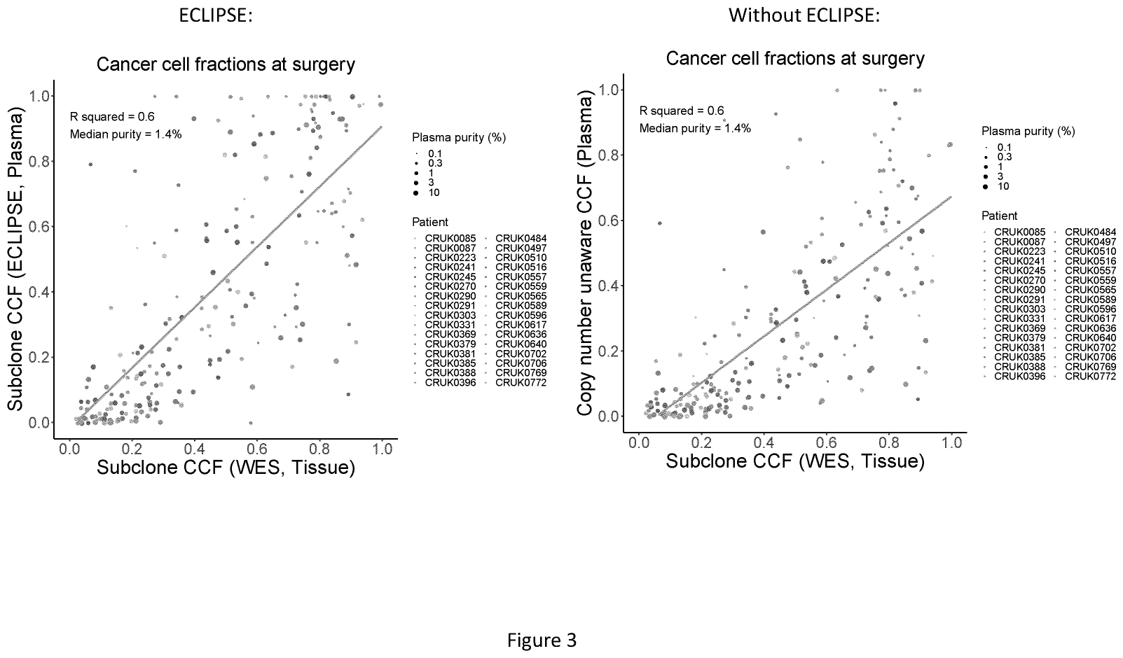

As shown in FIG. 3, the present inventors compared CCFs measured in plasma taken before surgery (y-axis) and measured using multiregional tissue sequencing at surgery (x-axis). The left-hand panel shows the data where the ECLIPSE tool of the present invention was used to estimate CCFs from the plasma samples. The right-hand panel shows the data where ECLIPSE was not used (i.e. CCF was estimated by VAF only, i.e. the mean VAF for each subclone divided by the mean VAF of the clonal cluster) in the estimation of CCFs from the plasma samples (i.e. copy number unaware CCF). The scatter plot points are individual patent samples and their size of the scatter plot points is proportional to the plasma purity (see inset scale from 0.1% purity to 10% purity). The present inventors found that there was strong correlation between the plasma-derived CCF estimate and the multiregional tissue-derived CCF estimate, suggesting that generally clone size or the number of cells in each clone has a strong influence on the amount of ctDNA that is released and validating ECLIPSE as a method for CCF estimation. Moreover, the present inventors found that without ECLIPSE there was a systemic bias towards lower CCFs in the plasma, likely due to a lack of copy number correction. Clonal mutations tend to occur more often before genome doubling and hence be at a higher copy number and hence a higher VAF. Without wishing to be bound by any particular theory, the present inventors believe that the outliers on the left may be caused by either differences in cfDNA shedding per cell or sampling bias in the primary tumour tissue sampling.

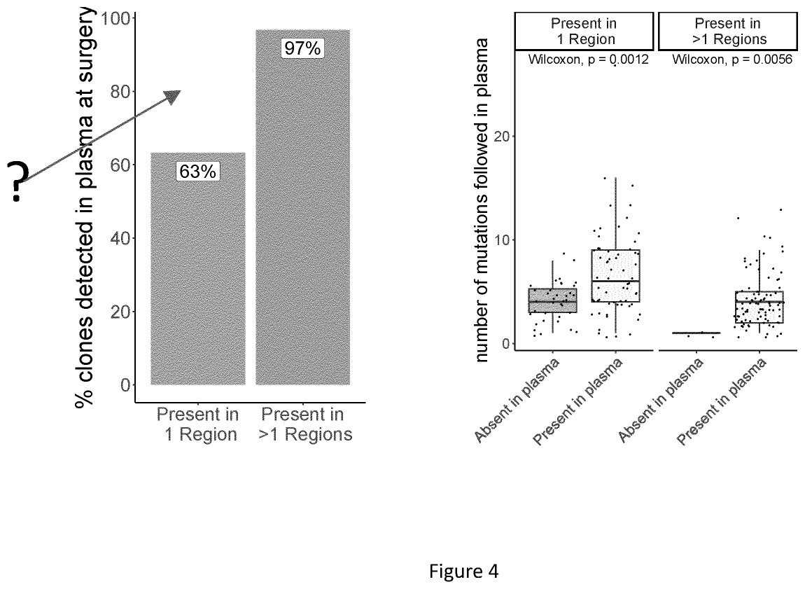

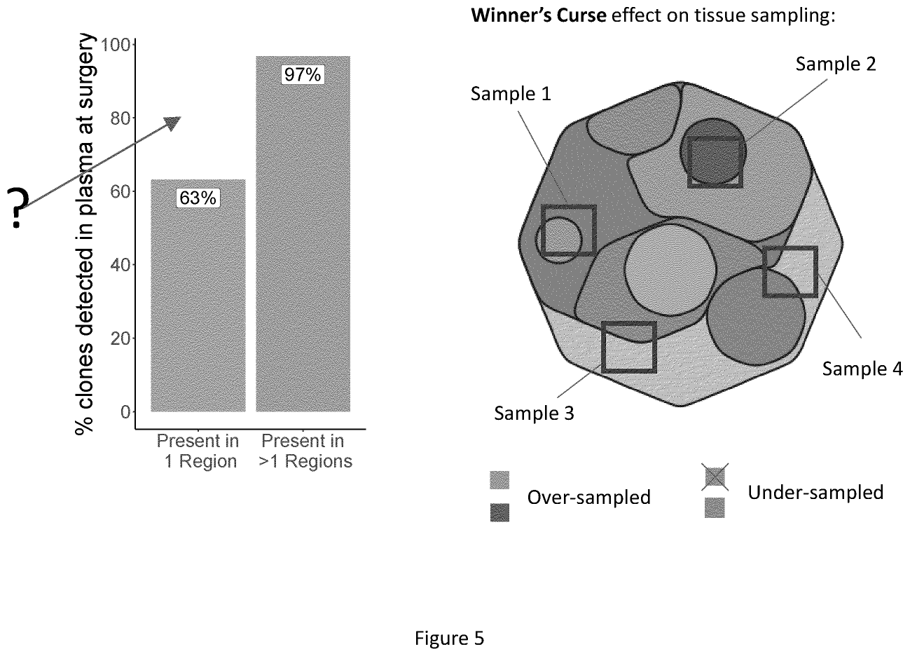

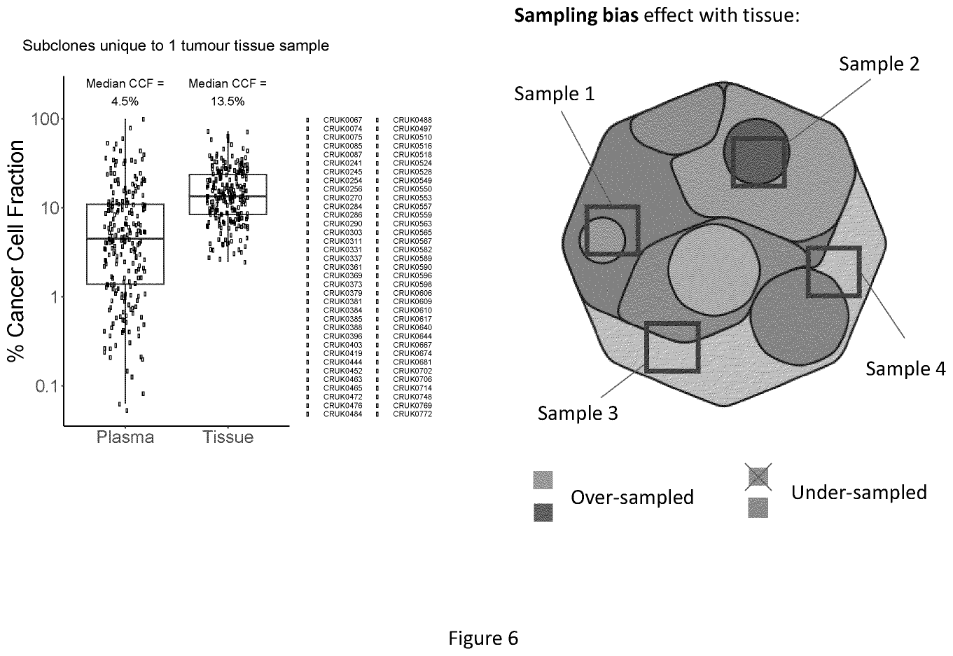

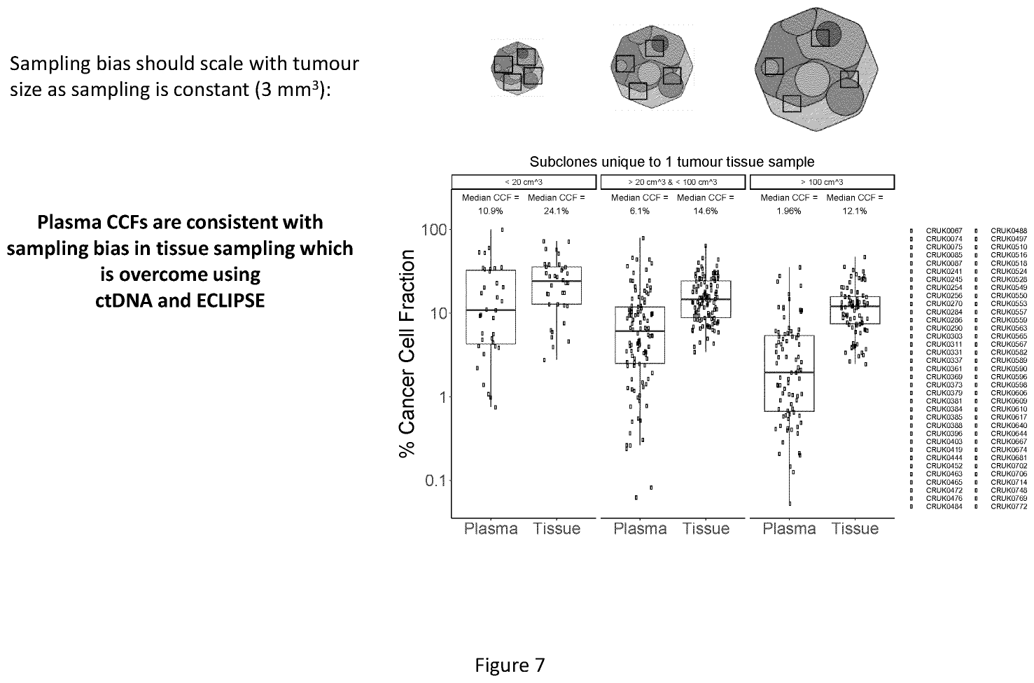

As shown in FIG. 4, the present inventors also assessed the ability to detect subclones at any cancer cell fraction in plasma samples. In this dataset it was estimated that the method had the power to detect subclones at 10% CCF in samples of 0.2 purity. A very high detection rate in plasma was found when clones were present across multiple samples in the primary tumour. However, if a clone was unique to a single sample there was a lower rate of detection. We found in the sample of the small minority of subclones missed which were spread across several regions, we had only tracked single mutations in these subclones. Hence these mutations may be false positives or not in fact members of their assigned subclone. In sample-unique clones however, while there was generally a lower number of mutations followed there were many clones with a high number of tracked mutations and where the CCF in the tissue would suggest we would be able to detect them in plasma. As shown in FIG. 5, one explanation for this finding is that these single region subclones have been over-sampled due to sampling bias in the primary tumour tissue and are in fact much smaller than we estimate from tissue—making their detection difficult in plasma. This particular form of sampling bias is known as the “Winner's Curse” effect. As shown in FIG. 6, consistent with such sampling bias, in the 60% of subclones which we DO detect and that are unique to a single regions, we usually estimate their clone size (CCF) to be smaller in plasma than in the tissue. Referring to FIG. 7, another prediction we would make if this was due to sampling bias is that this effect would scale with tumour size. That is to say, in larger tumours we would be likely to have a smaller percentage of their mass captured for sequencing, which in turn would mean that we would expect a stronger sampling bias. This is indeed what is seen in the present data. In small tumours, where much of the tumour is sequenced we see almost no decrease in estimated clone size in plasma vs. that estimated from tissue sampling. On the other hand, in larger tumours over 100 cm³, we see a strong effect where we may be overestimating clone size in the tissue by a factor of 6. Without wishing to be bound by any particular theory, the present inventors believe that this finding is consistent with plasma sampling—where appropriately processed using the ECLIPSE tool—giving a MORE accurate reading of tumour heterogeneity and CCF estimates than that obtained by tissue sampling. This may be attributable to the fact that the plasma sampling derives signal from a much larger and more representative pool of cells across the entire tumour. In other words, the use of ctDNA liquid biopsy plus ECLIPSE is not only less invasive, but has the potential to provide a more accurate estimate of CCF than tissue sampling of the tumour.

Representative sampling using plasma allows accurate identification of clonal mutations for therapeutic targeting (e.g. neoantigens that may be targetable for immunotherapy and/or cell therapy). In view of the above finding that plasma sampling represents well the clonal composition of the tumour mass, the present inventors considered that it may be possible to use plasma sampling to more accurately resolve clonality where comprehensive tumour tissue region sampling is not possible—for example, in inoperable patients at diagnosis, before neoadjuvant therapy or at relapse where multiple sites of metastasis are involved. Currently in these situations a single sample would be used to genomic profile patients and in some cases select targets for therapeutics. However, such a single sample may be highly susceptible to sampling bias, potentially resulting in suboptimal treatment target selection.

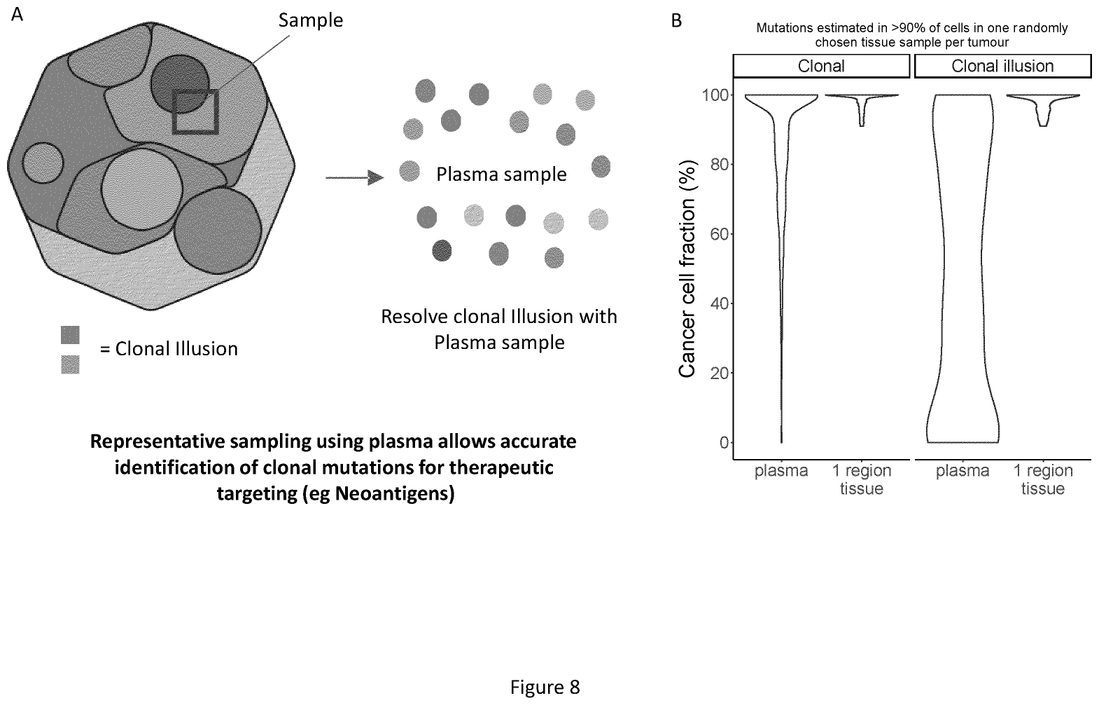

As is shown in FIG. 8A, a single sample usually leads to clonal illusions where mutations that are present in every cell of the sample taken are not present across the whole tumour and hence would be poor therapeutic targets. We simulated TRACERx into a single region dataset by randomly selecting a single sample for each patient and considering only the mutations which appear to be in every cell of that sample. We then split these apparently clonal mutations into those that were truly clonal across the whole tumour (see FIG. 8B, on the left) and those that had clonal illusions and which were, in fact, sub-clonal and not present in other samples (see FIG. 8B, on the right), plotting the estimated cancer cell fraction (CCF) on the y-axis. We saw in these cases that mutations with clonal illusion had much lower CCFs in the plasma, since the plasma sample better represents the proportion of cells across the whole tumour. This suggests that plasma samples could help determine whether a therapeutic target is truly clonal and hence worth therapeutically targeting.

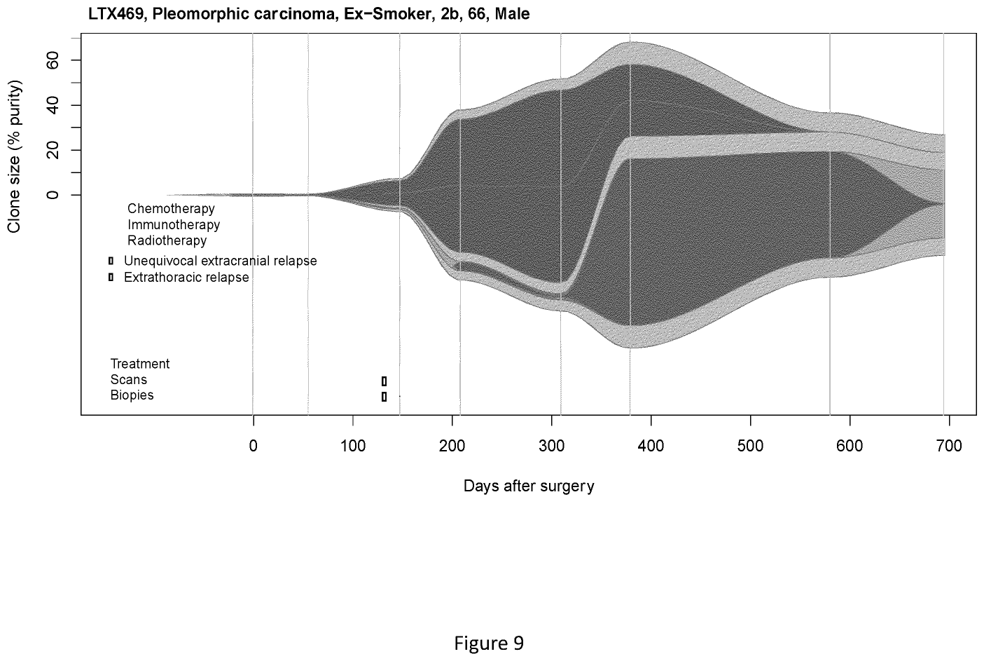

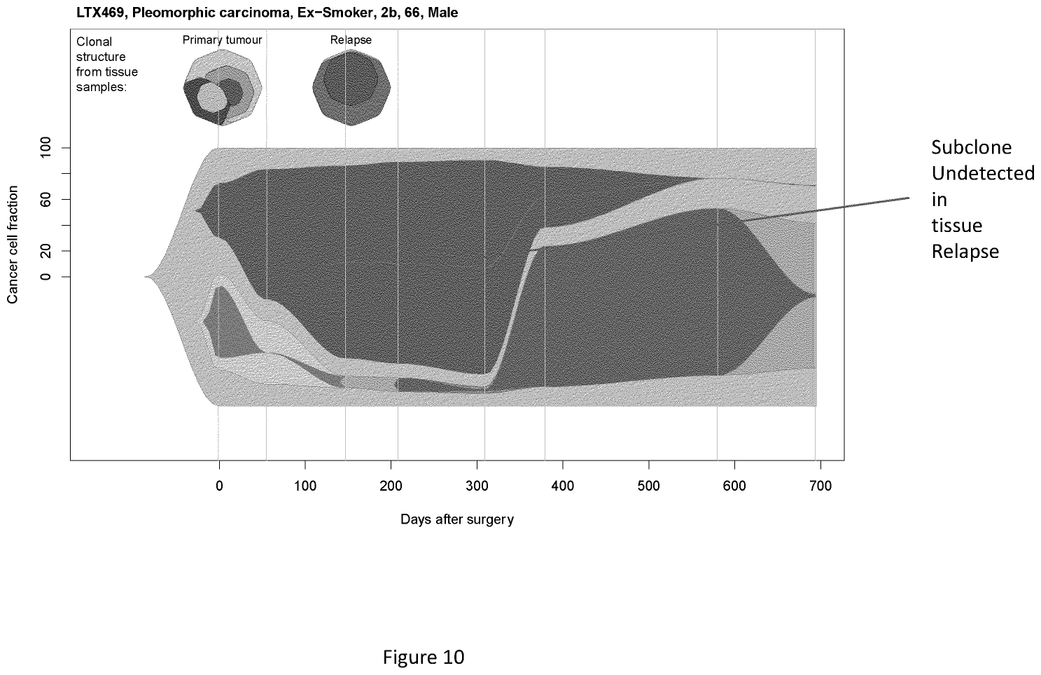

In FIG. 9, we seen an example of tumour dynamics tracked through time using ECLIPSE. In this plot the y-axis indicates the size of the different clones and the total tumour mass in the body. In this case we see two different clones dominate the tumour post-surgery. We also see that, after immunotherapy is applied to this patient, a shift in the fitness landscape where the red clone replicates more rapidly and out-competes the blue clone. If we represent this using cancer cell fractions—i.e. ignoring the total tumour mass we can now see (FIG. 10) that at earlier, low cellularity time points that the lineage of the red subclone is present in a high proportion of cells before surgery, but is initially out-competed by the blue subclone. However, the CCF of the blue clone then shrinks upon application of immune-oncology therapy (IO). We can also compare this to the clonal structure of relapsing tissue shown above this plot, where both lineages are represented in the primary tumour, detected using multi-region sequencing at surgery. Then, at later timepoints only the red and green, but not the blue lineage is detected, suggesting that the blue lineage is unique to other sites of disease not sampled using tissue biopsies but captured using ctDNA.

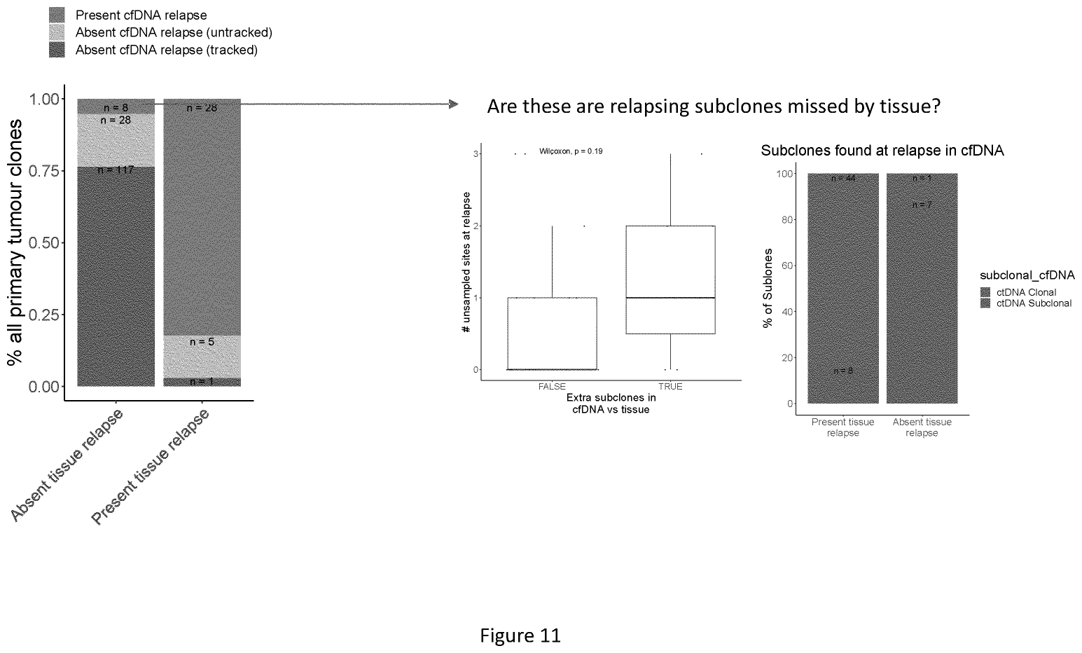

When the present inventors looked at the agreement between clones detected in ctDNA and in relapsing tissue profiles in TRACERx, for almost all (but 1) of the subclones tracked in ctDNA that those clones found in tissue were also detected in ctDNA. However, ctDNA also identified additional subclones providing a 28% increase in the number of subclones detected (see FIG. 11). These extra subclones may be present in unsampled metastatic sites and supporting this we found that patients with additional subclones discovered in ctDNA had a non-significant trend towards a higher number of unsampled sites and also these clones tended to be estimated to be in only a subset of the tumour cell using ctDNA-consistent with them being missed using tissue sampling.

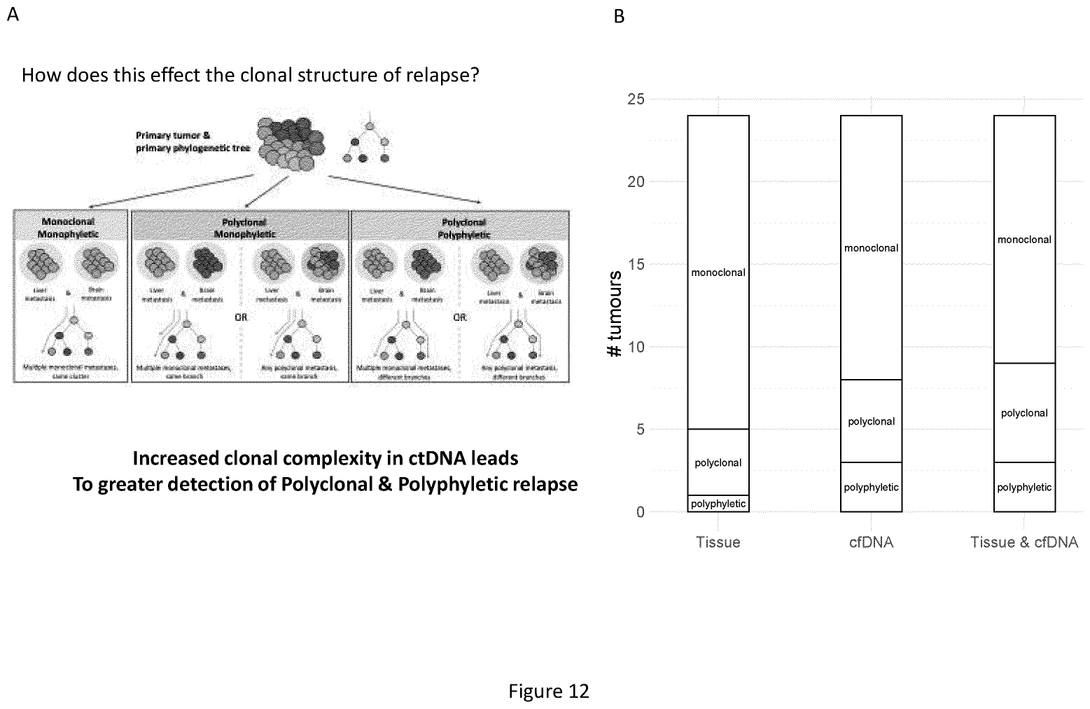

In TRACERx we have a strong interest in the modes of cancer metastasis (see FIG. 12A). Some patients present with monoclonal relapses, where only a single subclone seeded all the metastatic tissue. Some present polyclonal relapses, where multiple different clones seed the metastatic tissue or polyphyletic relapses, where multiple clones from multiple different branches on the phylogenetic tree seed the metastasis. We compared the seeding types detecting using ctDNA and detected using tissue sampling. We found that generally ctDNA provided a more clonally complex picture where more patients were classified as polyclonal or polyphyletic due to the additional subclones that were detected in the ctDNA. Despite this this, in agreement with the current data, the majority of patients seem to have monoclonal disease relapse (see FIG. 12B).

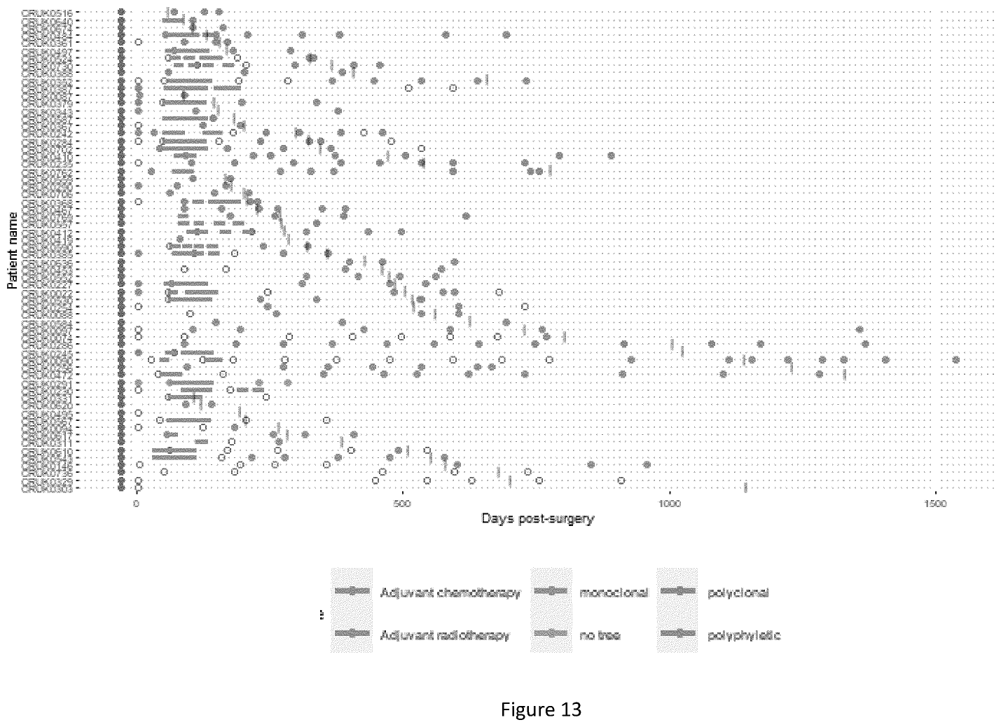

The plot in FIG. 13 shows each patient as a line with time running across the x-axis and samples represented as dots coloured according to the type of relapse detected. It can be seen that for most cases once the relapse type is detected it is consistent in subsequent samples and importantly the relapse type can be accurately detected often before clinical relapse (shown by the grey vertical lines) in the adjuvant, minimal residual disease (MRD) setting, despite the low ctDNA fractions in these samples.

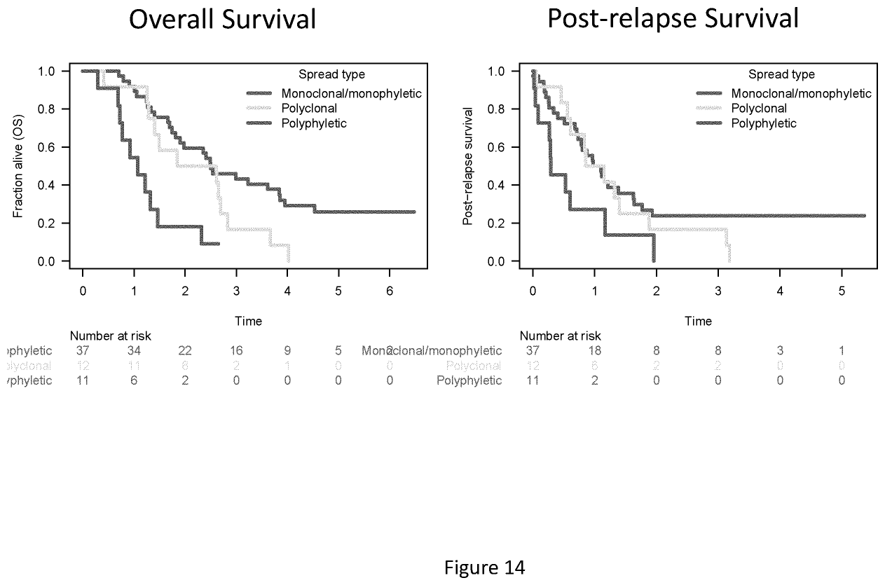

As shown in FIG. 14, more clonally complex tumours tend to have poor survival outcomes both from diagnosis and after relapse suggesting this allows further prognostic triaging after MRD detection.

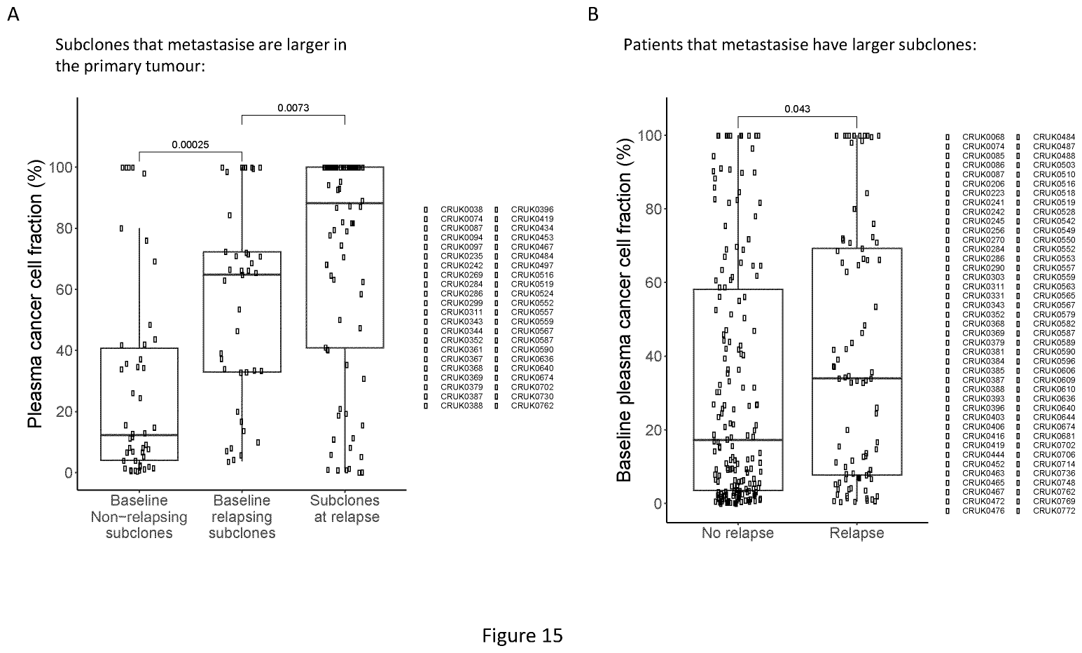

The use of plasma sampling coupled with ECLIPSE estimation of CCF, lead the present inventors to make an interesting insight related to subclone size and metastatic potential. A strong bias was found in the type of clones that seed metastasis (see FIG. 15B). In patients that go on to metastasize, we see that relapsing clones tend to have much higher cancer cell fractions in the plasma than those that do not. This suggests that it is the largest subclones that have undergone recent sub-clonal expanation which are the mostly likely to metastasize. We also saw that the distribution of clone sizes in metastasizing and non-metastasizing patients was also different—where the metastasizing patients tended to have more large subclones present in their tumour (see FIG. 15A).

### REFERENCES

A number of publications are cited above in order to more fully describe and disclose the invention and the state of the art to which the invention pertains. The entirety of each of these references is incorporated herein.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A computer-implemented method for estimating the cancer cell fraction (CCF) of at least one tumour-specific mutation in a subject, the method comprising:
(i) providing sequence data obtained from a sample comprising cell-free DNA, which includes circulating tumour DNA (ctDNA) from the subject, the sequence data comprising: the variant allele fraction (VAF), being equal to the total number of reads in the sample that show the tumour-specific mutation divided by the total number of reads (mutated and germline) at the location of the tumour-specific mutation;
(ii) providing sequence data obtained from a sample comprising DNA obtained from tumour tissue of the subject, the sequence data comprising: the multiplicity of said at least one tumour-specific mutation; and the copy number at the location of the tumour-specific mutation (CNₜᵤₘₒᵤᵣ);
(iii) providing the germline copy number at the location of the tumour-specific mutation (CNₙₒᵣₘₐₗ);
(iv) providing an estimate of the purity of said sample comprising cell-free DNA, the purity being the proportion of cells contributing to the sampled DNA which are tumour cells; and
(v) determining the estimate of CCF for the at least one tumour-specific mutation according to the formula:
$$CCF = VAF \frac{1}{multiplicity * Purity} ( Purity \times {CN}_{tumour} + ( 1 - Purity ) \times {CN}_{normal} )$$
wherein VAF is as provided in (i), multiplicity and CNₜᵤₘₒᵤᵣ are as provided in (ii), CNₙₒᵣₘₐₗ is as provided in (iii) and purity is as provided in (iv).

2. The method of claim 1, wherein providing an estimate of purity of said sample comprising cell-free DNA comprises
providing, for each of a plurality of further tumour-specific mutations that have previously been determined to be clonal mutations, the VAF of the further mutation in said sample comprising cell-free DNA, the multiplicity of the further mutation, the CNₜᵤₘₒᵤᵣ of the further mutation, and the CNₙₒᵣₘₐₗ at the location of the further mutation;
determining the mutation-specific purity of each of said plurality of further mutations according to the formula:
$$Purity = \frac{{CN}_{normal}}{\frac{multiplicity}{VAF} - {CN}_{tumour} + {CN}_{normal}}$$
and
estimating the purity of said sample by averaging the mutation-specific purity values of each of said plurality of further mutations.

3. The method of claim 1 or claim 2, wherein said at least one tumour-specific mutation comprises at least 2, 3, 4 or at least 5 tumour-specific mutations belonging to a single sub-clonal population of tumour cells.

4. The method of claim 3, wherein the estimated CCF for each of said tumour-specific mutations belonging to the single sub-clonal population are averaged to provide a CCF estimate for the sub-clonal population of tumour cells.

5. The method of any one of the preceding claims, wherein a correction for the background sequencing error is applied to estimate whether the number of reads in the sample that show the tumour-specific mutations from a given sub-clonal population of cells is likely to be genuine or due to sequencing error.

6. The method of claim 5, wherein a statistical test is applied to compare (i) the total number of reads in the sample that show the tumour-specific mutations from the sub-clonal population and (ii) the background sequencing error rate at the location of each of the tumour-specific mutations multiplied by the total number of reads at the location of each of the tumour-specific mutations.

7. The method of claim 6, wherein if the p-value of statistical test is greater than 0.05, the sub-clonal population of cells is considered not to be present in the sample.

8. The method of claim 6 or claim 7, wherein the statistical test is selected from the group consisting of: a binomial test, a Poisson test, a one sample Wilcoxon rank sum test, a chi-squared and a Fisher's exact test.

9. The method of any one of the preceding claims, wherein the sample comprising DNA obtained from tumour tissue of the subject is obtained at an earlier point in time than the sample comprising cell-free DNA.

10. The method of any one of the preceding claims, wherein sequence data is provided that has been obtained from multiple samples comprising cell-free DNA, which includes circulating tumour DNA (ctDNA), from the subject at different time points.

11. The method of claim 10, wherein the different time points comprise different time points during a course of treatment of the tumour.

12. The method of any one of the preceding claims, wherein the purity of the or each sample comprising cell-free DNA is 5% or lower, such as 4%, 3%, 2%, 1% or 0.5% or lower.

13. The method of any one of the preceding claims, wherein said at least one tumour-specific mutation gives rise to a suspected or known neoantigen and/or gives rise to a target for an anti-cancer therapy.

14. The method of any one of the preceding claims, further comprising providing to a user the determined CCF of at least one tumour-specific mutation and/or at least one clonal or sub-clonal tumour cell population, optionally wherein the determined CCF is displayed on a user interface or transmitted to the user via a network.

15. A method for estimating the cancer cell fraction (CCF) of at least one tumour-specific mutation in a subject, the method comprising:
providing a cfDNA-containing sample, which sample includes ctDNA, obtained from the subject;
sequencing DNA from said cfDNA-containing sample or from a library prepared from said cfDNA-containing sample to produce sequence data; and
performing the method of any one of claims 1 to 14 using said sequence data and thereby estimating the CCF of the at least one tumour-specific mutation in the subject.

16. The method of claim 15, wherein the method further comprises:
providing a sample comprising DNA obtained from tumour tissue of the subject;
sequencing DNA from said sample comprising DNA obtained from tumour tissue or from a library prepared from said sample comprising DNA obtained from tumour tissue to produce tumour tissue sequence data; and
analysing the tumour tissue sequence data produced to determine the multiplicity of said at least one tumour-specific mutation; and the copy number at the location of the tumour-specific mutation (CNₜᵤₘₒᵤᵣ).

17. A method for identifying at least one tumour-specific mutation, or a population of tumour cells harbouring said at least one tumour-specific mutation, in a subject as a potential therapeutic target, the method comprising:
performing the method of any one of claims 1 to 16 at least once to estimate the CCF of the at least one tumour-specific mutation or the population of cells harbouring said at least one tumour-specific mutation; and
selecting the at least one tumour-specific mutation or the population of cells harbouring said at least one tumour-specific mutation as a potential therapeutic target, provided that at least one of the following is true:
the CCF is estimated to be at least 0.3, 0.4, 0.5, 0.6, 0.7, 0.8, 0.9 or at least 0.95;
the CCF is estimated at at least two different time points and is found to be rising; and
the CCF is estimated before and after a treatment intervention for said tumour and the CCF is found to be declining following said treatment intervention.

18. A method for monitoring the clonal dynamics of a tumour and/or monitoring a treatment of the tumour, the method comprising:
performing the method of any one of claims 1 to 16 to estimate the CCF of the at least one tumour-specific mutation or the population of cells harbouring said at least one tumour-specific mutation at two or more time points for the same subject; and
tracking the estimated CCF at said two or more time points to monitor change in the CCF over time.

19. The method of claim 18, wherein the CCF of at least 2, 3, 4, 5, 6, 7, 8, 9, 10 or at least 20 tumour-specific mutations and/or the CCF of at least 2, 3, 4, 5, 6, 7, 8, 9, 10 or at least 20 clonally distinct populations of cells of said tumour are estimated.

20. The method according to any one of the preceding claims, wherein said at least one tumour-specific mutation is selected from the group consisting of: a single nucleotide variant (SNV), a multiple nucleotide variant (MNV), a deletion mutation, an insertion mutation, an indel mutation, a translocation, a missense mutation, a translocation, a fusion, a splice site mutation, or any other change in the genetic material of a tumour cell.

21. The method according to claim 20, wherein the at least one tumour-specific mutation results in the mutated DNA encoding a neoantigen and/or wherein the at least one tumour-specific mutation is or encodes a target for an anti-cancer therapy.

22. A method for treating a subject having a cancer, the method comprising:
performing the method of claim 21, wherein the estimated CCF of the at least one tumour-specific mutation indicates that the tumour-specific mutation is present in the present in the tumour at a level sufficient to render the tumour-specific mutation an effective target for therapy; and
administering an anti-cancer therapy that targets said tumour-specific mutation.

23. The method of claim 22, wherein at least one of the following is true:
the CCF is estimated to be at least 0.3, 0.4, 0.5, 0.6, 0.7, 0.8, 0.9 or at least 0.95;
the CCF is estimated at at least two different time points and is found to be rising; and
the CCF is estimated before and after administration of said anti-cancer therapy and the CCF is found to be declining following said administration.

24. The method of any one of the preceding claims, wherein:
the tumour in the subject has, or is suspected of having, metastasized;
the subject has had treatment aimed at surgical removal of one or more tumours;
the subject has been treated with one or more anti-cancer therapeutic agents; and/or
the subject has a cancer which has relapsed or the subject is suspected to be at risk of cancer relapse.

25. A system comprising:
a processor; and
a computer readable medium comprising instructions that, when executed by the processor, cause the processor to perform the steps of the method of any of claims 1 to 14.

26. One or more computer readable media comprising instructions that, when executed by one or more processors, cause the one or more processors to perform the steps of the method of any of claims 1 to 14.
